SEQUENCE LISTING (8 sequences)

SEQ ID NO: 1 - DNA, 4180 bp, Unknown
  Description of Unknown mRNA transcript encoding furin sequence
gcggggaagcagcagcggccaggatgaatcccaggtgctctggagctggatggtgaaggtcggcactcttcaccctcccgagccctgcccgtctcggccccatgcccccaccagtcagccccgggccacaggcagtgagcaggcacctgggagccgaggccctgtgaccaggccaaggagacgggcgctccagggtcccagccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccactgcccaccccctcaagccaatcccctccttgggcactttttaattcaccaaagtatttttttatcttgggactgggtttggaccccagctgggaggcaagaggggtggagactgcttcccatcctaccctcgggcccacctggccacctgaggtgggcccaggaccagctggggcgtggggagggccgtaccccaccctcagcaccccttccatgtggagaaaggagtgaaacctttagggcagcttgccccggccccggccccagccagagttcctgcggagtgaagaggggcagcccttgcttgttgggattcctgacccaggccgcagctcttgcccttccctgtccctctaaagcaataatggtcccatccaggcagtcgggggctggcctaggagatatctgagggaggaggccacctctccaagggcttctgcaccctccaccctgtcccccagctctggtgagtcttggcggcagcagccatcataggaagggaccaaggcaaggcaggtgcctccaggtgtgcacgtggcatgtggcctgtggcctgtgtcccatgacccacccctgtgctccgtgcctccaccaccactggccaccaggctggcgcagccaaggccgaagctctggctgaaccctgtgctggtgtcctgaccaccctcccctctcttgcacccgcctctcccgtcagggcccaagtccctgttttctgagcccgggctgcctgggctgttggcactcacagacctggagcccctgggtgggtggtggggaggggcgctggcccagccggcctctctggcctcccacccgatgctgctttcccctgtggggatctcaggggctgtttgaggatatattttcactttgtgattatttcactttagatgctgatgatttgtttttgtatttttaatgggggtagcagctggactacccacgttctcacacccaccgtccgccctgctcctccctggctgccctggccctgaggtgtgggggctgcagcatgttgctgaggagtgaggaatagttgagccccaagtcctgaagaggcgggccagccaggcgggctcaaggaaagggggtcccagtgggaggggcaggctgacatctgtgtttcaagtggggctcgccatgccgggggttcataggtcactggctctccaagtgccagaggtgggcaggtggtggcactgagcccccccaacactgtgccctggtggagaaagcactgacctgtcatgcccccctcaaacctcctcttctgacgtgccttttgcacccctcccattaggacaatcagtcccctcccatctgggagtccccttttcttttctaccctagccattcctggtacccagccatctgcccaggggtgccccctcctctcccatccccctgccctcgtggccagcccggctggttttgtaagatgctgggttggtgcacagtgatttttttcttgtaatttaaacaggcccagcattgctggttctatttaatggacatgagataatgttagaggttttaaagtgattaaacgtgcagactatgcaaaccag

SEQ ID NO: 2 - RNA, 245 nt, Artificial Sequence
  Description of Artificial Sequence Synthetic polynucleotide
gauccugcuguugacagugagcgcggagaaaggagugaaaccuuagugaagccacagauguaagguuucacuccuuucuccuugccuacugccucggaagcagcucacuacauuacucagcuguugacagugagcgcggagaaagauaugaaaccuuagugaagccacagauguaagguuucacuccuuucuccuugccuacugccucggaagcuuaauaaaggaucuuuuauuuucauuggauc

SEQ ID NO: 3 - RNA, 21 nt, Artificial Sequence
  Description of Artificial Sequence Synthetic oligonucleotide
ggagaaaggagugaaaccuua

SEQ ID NO: 4 - RNA, 21 nt, Artificial Sequence
  Description of Artificial Sequence Synthetic oligonucleotide
uaagguuucacuccuuucucc

SEQ ID NO: 5 - RNA, 21 nt, Artificial Sequence
  Description of Artificial Sequence Synthetic oligonucleotide
ggagaaagauaugaaaccuua

SEQ ID NO: 6 - RNA, 15 nt, Artificial Sequence
  Description of Artificial Sequence Synthetic oligonucleotide
gugaagccacagaug

SEQ ID NO: 7 - DNA, 5140 bp, Artificial Sequence
  Description of Artificial Sequence Synthetic polynucleotide
ggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctaaaaaaaaaaaaaaaaaaaaaaacggaattcacgtgggcccggtaccgtatactctagaagatctggcagcggagagggcagaggaagtcttctaacatgcggtgacgtggaggagaatcccggccctaggatgtctagagcggccgcggatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctatt

SEQ ID NO: 8 - RNA, 223 nt, Artificial Sequence
  Description of Artificial Sequence Synthetic polynucleotide
gauccugcuguugacagugagcgcggagaaaggagugaaaccuuagugaagccacagauguaagguuucacuccuuucuccuugccuacugccucggaagcagcucacuacauuacucagcuguugacagugagcgcggagaaagauaugaaaccuuagugaagccacagauguaagguuucacuccuuucuccuugccuacugccucggaagcuuaauaaag

(19) United States
(12) Patent Application Publication
(10) Pub. No.: US 2024/0189450 A1
(43) Pub. Date: Jun. 13, 2024

(54) METHODS FOR TREATING CANCERS

(71) Applicant: GRADALIS, INC., Carrollton, TX (US)

(72) Inventors: John Nemunaitis, Carrollton, TX (US); Ernest Bognar, Carrollton, TX (US); Elyssa Sliheet, Carrollton, TX (US); Molly Robinson, Carrollton, TX (US); Susan Morand, Carrollton, TX (US); Laura Nejedlik, Carrollton, TX (US)

(73) Assignee: GRADALIS, INC., Carrollton, TX (US)

(21) Appl. No.: 18/285,214

(22) PCT Filed: Mar. 9, 2022
(86) PCT No.: PCT/US2022/019492

§ 371 Date: Sep. 29, 2023

Related U.S. Application Data
(60) Provisional application No. 63/159,341, filed on Mar. 10, 2021.

(51) Int. Cl. A61K 48/00; A61K 45/06; C12Q 1/6886

(52) U.S. Cl. CPC A61K 48/005; A61K 45/06; C12Q 1/6886; C12Q 2600/106; C12Q 2600/156

(57) ABSTRACT

Disclosed herein are methods for predicting the responsiveness of a cancer in a cancer patient and methods for treating the cancer by identifying the genotype of one or more genes in the patient.

### CROSS-REFERENCE TO RELATED APPLICATION

This application claims priority to U.S. Provisional Application No. 63/159,341, filed Mar. 10, 2021, the disclosure of which is hereby incorporated by reference in its entirety for all purposes.

### BACKGROUND

When the homologous recombination (HR) pathway is working properly, DNA can be repaired effectively and is error free, maintaining genomic stability. When the HR pathway is disrupted by gene mutations, promoter methylation, or unknown causes, the HR pathway stops working, leading to genomic instability or homologous recombination deficiency (HRD). The BRCA gene plays a role in DNA repair via HR, and mutations of this gene can lead to HRD.

### BRIEF SUMMARY

In one aspect, the disclosure features a method for predicting the responsiveness of a cancer in a cancer patient to a cancer treatment, comprising determining the genotypes of BRCA1 and/or BRCA2 in a sample from the cancer patient, wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2; and wherein a determination of one or more of the following genotypes: (1) BRCA1wt, (2) BRCA2 wt, and (3) BRCA1wt and BRCA2 wt, indicates that the cancer patient is responsive to the cancer treatment.

In some embodiments of this aspect, the method comprises determining the genotypes of two or more genes selected from the group consisting of BRCA1, BRCA2, TP53, PIK3CA, NF1, ARID1A, MYCNOS, and MUTYH, and wherein one of the two or more genes is BRCA1 or BRCA2.

In some embodiments of this aspect, a determination of one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt, indicates that the cancer patient is responsive to the cancer treatment.

In some embodiments of this aspect, the method comprises determining the genotypes of three genes, and wherein two of the three genes are BRCA1 and BRCA2. In some embodiments, a determination of one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt, indicates that the cancer patient is response to the cancer treatment.

In another aspect, the disclosure features, a method for predicting the responsiveness of a cancer in a cancer patient to a cancer treatment, comprising determining the genotypes of a first gene and a second gene, in a sample from the cancer patient, wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2; wherein the first gene is ARID1A and the second gene is selected from the group consisting of CTNNB1, MUTYH, NF1, PIK3CA, and UVSSA; and wherein a determination of one or more of the following pairs of genotypes: CTNNB1wt and ARID1Am, MUTYHwt and ARID1Am, NF1wt and ARID1Am, PIK3CAwt and ARID1Am, and UVSSAwt and ARID1Am, indicates that the cancer patient is responsive to the cancer treatment.

In some embodiments of the methods described herein, the cancer patient is identified as homologous recombination proficient. In certain embodiments, the method, upon the determination of genotype(s) that indicates responsiveness of the cancer patient to the cancer treatment, further comprises treating the cancer patient with the cancer treatment.

In some embodiments, the sample is a biopsy sample. In particular embodiments, the biopsy sample is a biopsy sample of the tumor cells or a biopsy sample of circulating tumor cells.

In another aspect, the disclosure provides a method for treating a cancer in a cancer patient in need thereof, the method comprising administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, wherein the cancer patient comprises one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt.

In another aspect, the disclosure features a method for treating a cancer in a cancer patient in need thereof, the method comprising administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, wherein the cancer patient comprises one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt.

In another aspect, the disclosure features a method for treating a cancer in a cancer patient in need thereof, the method comprising administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, wherein the cancer patient comprises one or more of the following pairs of genotypes: CTNNB1wt and ARID1Am, MUTYHwt and ARID1Am, NF1 wt and ARID1Am, PIK3CAwt and ARID1Am, and UVSSAwt and ARID1Am, indicates that the cancer patient is responsive to the cancer treatment.

In some embodiments of methods described herein, the cancer patient is identified as homologous recombination proficient.

In another aspect, the disclosure provides a method for treating a cancer in a cancer patient in need thereof, the method comprising: 1) genotyping the cancer patient to identify genotypes comprising BRCA1wt and/or BRCA2 wt in a sample from the cancer patient; 2) administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, to thereby treat the cancer patient.

In some embodiments of this aspect, step 1) further comprises one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt.

In some embodiments of this aspect, step 1) further comprises one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt.

In another aspect, the disclosure provides a method for treating a cancer in a cancer patient in need thereof, the method comprising: 1) genotyping the cancer patient to identify one or more of the following pairs of genotypes: CTNNB1wt and ARID1Am, MUTYHwt and ARID1Am, NF1wt and ARID1Am, PIK3CAwt and ARID1Am, and UVSSAwt and ARID1Am, in a sample from the cancer patient; 2) administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, to thereby treat the cancer patient.

In some embodiments, the sample is a biopsy sample, e.g., a biopsy sample of the tumor cells or a biopsy sample of circulating tumor cells.

In some embodiments of the methods described herein, the GM-CSF is a human GM-CSF sequence. In some embodiments, the expression vector further comprises a promoter, e.g., a cytomegalovirus (CMV) mammalian promoter. In some embodiments, the expression vector further comprises a CMV enhancer sequence and a CMV intron sequence. In some embodiments, the expression vector further comprises a nucleic acid sequence encoding a picomaviral 2A ribosomal skip peptide between the first and the second nucleic acid inserts. In certain embodiments, the expression vector is within an autologous cancer cell that is transfected with the expression vector. In certain embodiments, the autologous cancer cell is administered to the individual as a dose of about 1×106 cells to about 5×107 cells. In particular embodiments, the autologous cancer cell is administered to the individual once a month. In some embodiments, the autologous cancer cell is administered to the individual from 1 to 12 months. In some embodiments, the autologous cancer cell is administered to the cancer patient by intradermal injection. In some embodiments of the methods described herein, the first insert and the second insert are operably linked to the promoter.

In some embodiments, the cancer is an HRD-negative, wild-type BRCA1/2 cancer. In certain embodiments, the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer. In particular embodiments, the solid tumor cancer is selected from the group consisting of endometrial cancer, biliary cancer, bladder cancer, liver hepatocellular carcinoma, gastric/esophageal cancer, ovarian cancer, melanoma, breast cancer, pancreatic cancer, colorectal cancer, glioma, non-small-cell lung carcinoma, prostate cancer, cervical cancer, kidney cancer, thyroid cancer, a neuroendocrine cancer, small cell lung cancer, a sarcoma, head and neck cancer, brain cancer, clear cell renal cell carcinoma, skin cancer, endocrine tumor, thyroid cancer, tumor of unknown origin, and a gastrointestinal stromal tumor. In particular embodiments, the cancer is ovarian cancer. In particular embodiments, the cancer is breast cancer. In particular embodiments, the cancer is melanoma. In particular embodiments, the cancer is lung cancer.

In some embodiments of the methods described herein, the cancer is ovarian cancer and the method prevents or delays relapse of a substantially eradicated ovarian cancer. In certain embodiments, the substantially eradicated ovarian cancer is Stage III or Stage IV ovarian cancer.

In some embodiments of the methods described herein, the cancer patient received an initial therapy. In certain embodiments, the initial therapy comprises debulking surgery, chemotherapy, or the combination thereof. In certain embodiments, the chemotherapy comprises administering a platinum-based drug and a taxane. In certain embodiments, the the platinum-based drug comprises carboplatin. In certain embodiments, the taxane comprises paclitaxel.

In some embodiments of the methods described herein, the methods further comprise administering an additional therapeutic agent. In certain embodiments, the additional therapeutic agent is a member selected from the group consisting of an angiogenesis inhibitor, a PARP inhibitor, and a checkpoint inhibitor to the individual.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a visual display of the mutation profiles of all patients.

FIG. 2 shows a bar chart displaying the degrees of each gene in the overall set. Genes with a black bar correspond to genes included in the close Distance Matrix. These genes allow for more protein-protein interactions in the construction of the STRING network. Gray bars are genes from the overall gene set that were not represented in the Distance Matrix.

FIG. 3 shows a Distance Matrix. The distance matrix only includes 23 genes as this is a subset of the network that is fully connected. Short distances indicate a high degree of interaction between the genes. Large distances indicate less interaction between a gene pair. The diagonal represents a gene's interaction with itself, and it is equal to 0.

FIG. 4 shows RFS from randomization of TP53m-BRCAwt population (HR=0.44, p=0.01).

FIGS. 5A and 5B show RFS (HR=0.26, p=0.001) (FIG. 5A) and OS (HR=0.33, p=0.02) (FIG. 5B) from time of randomization for TP53m-BRCAwt and HRP population.

FIG. 6 is a schematic showing the bi-shRNA^{furin} (SEQ ID NO:2) comprising two stem-loop structures each with a miR-30a loop; the first stem-loop structure has complete complementary guiding strand and passenger strand, while the second stem-loop structure has three basepair (bp) mismatches at positions 9 to 11 of the passenger strand.

### DETAILED DESCRIPTION

Disclosed herein, in certain embodiments, are methods of treating a cancer in a cancer patient in need thereof by: 1) genotyping the cancer patient to identify genotypes comprising BRCA1wt and/or BRCA2 wt in a sample from the cancer patient; 2) administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, to thereby treat the cancer patient. In other embodiments, the methods comprise genotyping the cancer patient to identify one or more of the following pairs of genotypes: CTNNB1wt and ARID1Am, MUTYHwt and ARID1Am, NF1wt and ARID1Am, PIK3CAwt and ARID1Am, and UVSSAwt and ARID1Am.

### Certain Definitions

Unless defined otherwise, all technical and scientific terms used herein have the same meaning as is commonly understood by one of skill in the art to which the claimed subject matter belongs.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

The terms “effective amount” or “therapeutically effective amount,” as used herein, refer to a sufficient amount of an agent or a compound being administered which will relieve to some extent one or more of the symptoms of the disease or condition being treated or prevent the onset or recurrence of the one or more symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the expression vector or autologous cancer cell vaccine required to provide a clinically significant decrease in disease symptoms without undue adverse side effects. In another example, an “effective amount” for therapeutic uses is the amount of the expression vector or autologous cancer cell vaccine as disclosed herein required to prevent a relapse of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some embodiments, “an effective amount” or “a therapeutically effective amount” varies from subject to subject, due to variation in metabolism of the expression vector or autologous cancer cell vaccine, age, weight, general condition of the subject, the condition being treated, the severity of the condition being treated, and the judgment of the prescribing physician.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and autologous tumor cell vaccines provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing relapse of the disease or condition, or inhibiting the symptoms of the disease or condition either prophylactically and/or therapeutically. In a non-limiting example, for prophylactic benefit, an expression vector or autologous cancer cell vaccine composition disclosed herein is administered to an individual at risk of developing a particular disease or condition, predisposed to developing a particular disease or condition, or to an individual previously suffering from and treated for the disease or condition.

As used herein, the term “responsiveness” or “response” refers to a positive reaction or change of a disease towards a therapy, e.g., a cancer's positive reaction towards a cancer therapy. A cancer's responsiveness to a cancer therapy can be measured by assessing the appearance, severity, and/or frequency of the symptoms of the cancer. In some embodiments, a cancer's responsiveness to a cancer therapy can be measured by the cancer patient's overall survival or relapse-free survival.

As used herein, the term “transfection” refers to the introduction of foreign DNA into eukaryotic cells. In some embodiments, transfection is accomplished by any suitable means, such as for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some embodiments, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some embodiments, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some embodiments, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some embodiments, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some embodiments, the term “nucleic acid” or “nucleic acid molecule” also includes so-called “peptide nucleic acids,” which comprise naturally-occurring or modified nucleic acid bases attached to a polyamide backbone. In some embodiments, nucleic acids are single stranded or double stranded.

As used herein, the term “expression vector” refers to nucleic acid molecules encoding a gene that is expressed in a host cell. In some embodiments, an expression vector comprises a transcription promoter, a gene, and a transcription terminator. In some embodiments, gene expression is placed under the control of a promoter, and such a gene is said to be “operably linked to” the promoter. In some embodiments, a regulatory element and a core promoter are operably linked if the regulatory element modulates the activity of the core promoter. As used herein, the term “promoter” refers to any DNA sequence which, when associated with a structural gene in a host yeast cell, increases, for that structural gene, one or more of 1) transcription, 2) translation, or 3) mRNA stability, compared to transcription, translation or mRNA stability (longer half-life of mRNA) in the absence of the promoter sequence, under appropriate growth conditions.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “homologous recombination deficiency-positive,” “HRD-positive,” and “HRD” are used interchangeably and they refer to the status that HR is deficient. Conversely, the term “homologous recombination deficiency-negative,” “HRD-negative,” “homologous recombination proficient,” and “HRP” are used interchangeably, and they refer to the status that HR is not deficient.

As used herein, if a gene name is followed by “wt,” it means that the genotype of the gene is wild-type.

As used herein, if a gene name is followed by “m,” it means that the genotype of the gene is mutated.

### Methods of Treating Cancer

Vigil® is an autologous tumor DNA immunotherapy transfected with a plasmid encoding GM-CSF and bifunctional short hairpin RNA inhibitor against furin. Furin is an enzyme essential for cleaving TGF-beta into its active form [19]. Vigil® was designed to enhance the immune system's potency against cancer in 3 ways: first, Vigil® introduces the individual tumor neoantigen repertoire to the immune system. Second, Vigil® enhances differentiation and activation of immune cells via GM-CSF, a cytokine important to immune activation at both the peripheral and marrow levels. Finally, Vigil® inhibits cancer expressing TGF-beta, thereby decreasing immunosuppressive activity of TGF-beta. Functional immune activation of Vigil® in correlation with clinical benefit has been demonstrated via ELISPOT assay [20, 21]. Moreover, Vigil® appears to increase CD3+/CD8+ T cell circulation in advanced solid tumor patients and expands MHC-II expression activity via NanoString analysis in correlation with clinical benefit [22, 23]. Safety and efficacy of Vigil® has been evaluated in several tumor types in addition to ovarian cancer [20, 21, 24-28].

A randomized double-blind placebo-controlled study (VITAL trial) of Vigil® versus placebo as maintenance therapy for frontline Stage III/IV ovarian cancer recently demonstrated clinical benefit from randomization in recurrence free survival (RFS) and overall survival (OS) in patients with BRCAwt tumors [19]. We hypothesize that intact DNA repair mechanisms of BRCAwt, HRP ovarian cancer may be important for Vigil® efficacy, possibly related to higher degree of clonal versus subclonal neoantigens available for anticancer immune stimulation as opposed to poor DNA repair as involved in BRCA mutant (BRCAm) and/or HRD molecular profile [29, 30].

The disclosure describes molecular analysis of genomic variant data in patients receiving Vigil® or placebo in a randomized double-blind trial to treat frontline Stage III/IV ovarian cancer. The disclosure identifies significant genomic variants, meaningful variant combinations, and relevant genes at the intersection or “hub” of ovarian cancer pathways.

The disclosure provides methods for predicting the responsiveness of a cancer in a cancer patient to a cancer treatment, comprising determining the genotypes of BRCA1 and/or BRCA2 in a sample from the cancer patient, wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2; and wherein a determination of one or more of the following genotypes: (1) BRCA1wt, (2) BRCA2 wt, and (3) BRCA1wt and BRCA2 wt, indicates that the cancer patient is responsive to the cancer treatment.

In some embodiments, the methods comprise the determination of the genotypes of two genes. When the first gene is BRCA1, the method further comprises the determination of the genotype of a second gene selected from the group consisting of BRCA2, TP53, PIK3CA, NF1, ARID1A, MYCNOS, and MUTYH.

In some embodiments, the methods comprise the determination of the genotypes of two genes. When the first gene is BRCA2, the method further comprises the determination of the genotype of a second gene selected from the group consisting of BRCA1, TP53, PIK3CA, NF1, ARID1A, MYCNOS, and MUTYH.

The cancer in the cancer patient is predicted to be responsive to the cancer treatment if the patient is determined to have one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt.

In certain embodiments, the methods comprise determining the genotypes of three genes in the cancer patient. In some embodiments, two of the three genes are BRCA1 and BRCA2. In some embodiments, the cancer in the cancer patient is predicted to be responsive to the cancer treatment if the patient is determined to have one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt, indicates that the cancer patient is response to the cancer treatment.

The disclosure also provides methods for predicting the responsiveness of a cancer in a cancer patient to a cancer treatment, comprising determining the genotypes of a first gene and a second gene, in a sample from the cancer patient, wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2.

In particular embodiments, the disclosure also provides methods for predicting the responsiveness of a cancer in a cancer patient to a cancer treatment, comprising determining the genotypes of a first gene and a second gene, in a sample from the cancer patient, wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2; wherein the first gene is ARID1A and the second gene is selected from the group consisting of CTNNB1, MUTYH, NF1, PIK3CA, and UVSSA; and wherein a determination of one or more of the following pairs of genotypes: CTNNB1wt and ARID1Am, MUTYHwt and ARID1Am, NF1wt and ARID1Am, PIK3CAwt and ARID1Am, and UVSSAwt and ARID1Am, indicates that the cancer patient is responsive to the cancer treatment.

The cancer patient may be identified as homologous recombination proficient. Once the determination of genotype(s) indicates responsiveness of the cancer in the cancer patient to the cancer treatment, the method can further comprise treating the cancer patient with the cancer treatment.

In addition to methods for predicting the responsiveness of the cancer to the cancer treatment, the disclosure also provides methods for treating a cancer in a cancer patient in need thereof by administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, in which the cancer patient comprises one or more of the sets of genotypes as shown in Table A below.

TABLE A

| | | |
|---|---|---|
| TP53m and BRCA1wt | BRCA2wt and MYCNOSwt | BRCA1wt, BRCA2wt, and |
| | | MUTYHwt |
| TP53m and BRCA2wt | BRCA2wt and MUTYHwt | BRCA1wt, BRCA2wt, and |
| | | ARID1Awt |
| BRCA1wt and PIK3CAwt | PRKNwt and BRCA2wt | CTNNB1wt and ARID1Am |
| BRCA1wt and NF1wt | RAD51Cwt and BRCA2wt | NF1wt and ARID1Am |
| BRCA1wt and ARID1Awt | RUNX1wt and BRCA2wt | PIK3CAwt and ARID1Am |
| BRCA1wt and MYCNOSwt | TP53m, BRCA1wt, and | MUTYHwt and ARID1Am |
| | BRCA2wt | |
| BRCA1wt and MUTYHwt | HTR2Cwt and BRCA2wt | UVSSAwt and ATRXwt |
| BRCA2wt and PIK3CAwt | BRCA1wt, BRCA2wt, and | TP53m and HTR2Cm |
| | PIK3CAwt | |
| BRCA2wt and NF1wt | BRCA1wt, BRCA2wt, and | UVSSAwt and ARID1Am |
| | NF1wt | |
| BRCA2wt and ARID1Awt | BRCA1wt, BRCA2wt, and | TP53m and ERCC2wt |
| | MYCNOSwt | |

In certain embodiments, the cancer patient receiving the cancer treatment (e.g., an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2) has the genotypes: BRCA1wt, BRCA2 wt, or BRCA1wt and BRCA2 wt.

In certain embodiments, the cancer patient receiving the cancer treatment (e.g., an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2) has one of the following sets of genotypes: TP53m and BRCA1wt; TP53m and BRCA2 wt; or TP53m, BRCA1wt, and BRCA2 wt.

In certain embodiments, the cancer patient receiving the cancer treatment (e.g., an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2) has the genotype ARID1Am. In some embodiments, the cancer patient has a pair of genotypes in which one genotype is ARID1Am. In some embodiments, the cancer patient has one of the following sets of genotypes: CTNNB1 wt and ARID1Am; HTR2Cwt and ARID1Am; MUTYHwt and ARID1Am; NF1 wt and ARID1Am; PIK3CAwt and ARID1Am; and UVSSAwt and ARID1Am.

In any of the embodiments described above, in certain embodiments of the methods, the cancer patient is identified as homologous recombination proficient.

The disclosure also provides methods for treating a cancer in a cancer patient in need thereof, the method comprising: 1) genotyping the cancer patient to identify genotypes comprising BRCA1wt and/or BRCA2 wt in a sample from the cancer patient; 2) administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, to thereby treat the cancer patient. In certain embodiments, step 1) comprises genotyping the cancer patient to identify one or more sets of the genotypes as provided in Table A.

In particular, in certain embodiments, step 1) comprises genotyping the cancer patient to identify one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt. In particular, in certain embodiments, step 1) comprises genotyping the cancer patient to identify one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt.

In particular embodiments, the disclosure provides methods for treating a cancer in a cancer patient in need thereof, the method comprising: 1) genotyping the cancer patient to identify one or more of the following sets of genotypes: TP53m and BRCA1wt; TP53m and BRCA2 wt; or TP53m, BRCA1wt, and BRCA2 wt, in a sample from the cancer patient; 2) administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, to thereby treat the cancer patient.

In particular embodiments, the disclosure provides methods for treating a cancer in a cancer patient in need thereof, the method comprising: 1) genotyping the cancer patient to identify the genotype ARID1Am and the genotype of one or more other genes, in a sample from the cancer patient; 2) administering to the cancer patient an expression vector comprising: a) a first insert comprising a nucleic acid sequence encoding a Granulocyte Macrophage Colony Stimulating Factor (GM-CSF) sequence; and b) a second insert comprising a sequence according to SEQ ID NO:2, to thereby treat the cancer patient. In certain embodiments, step 1) comprises genotyping the cancer patient to identify one or more of the following sets of genotypes: CTNNB1 wt and ARID1Am, MUTYHwt and ARID1Am, NF1 wt and ARID1Am, PIK3CAwt and ARID1Am, and UVSSAwt and ARID1Am, in a sample from the cancer patient.

In some embodiments, one or more available sequencing techniques can be used to determine the genotype of one or more genes in the cancer patient. In some embodiments, the sequencing comprises Sanger sequencing or next generation sequencing. In some embodiments, the next generation sequencing comprises massively parallel sequencing. In some embodiments, determining the genotypes comprises hybridization of nucleic acid extracted from the individual to an array. In some embodiments, the array is a microarray. In some embodiments, determining the genotypes comprises array comparative genomic hybridization of nucleic acid extracted from the individual.

In the methods described herein, in some embodiments, a sample can be a tissue sample. In some embodiments, a sample can be a biopsy sample from the patient, such as a biopsy sample of the tumor cells or a biopsy sample of circulating tumor cells.

In some embodiments, to characterize whether an individual is HRD-positive or HRD-negative, an HRD score can be determined. In some embodiments, an HRD score can be calculated based on scores for the loss of heterozygosity (LOH), telomeric allelic imbalance (TAI), and large-scale state transitions (LSTs). In some embodiments, the LOH is indicated by the presence of a single allele. In some embodiments, the LOH is defined as the number of chromosomal loss of heterozygosity regions longer than 15 Mb. In some embodiments, the TAI is indicated by a discrepancy in the 1 to 1 allele ratio at the end of the chromosome. In some embodiments, the LSTs are indicated by transition points between regions of abnormal and normal DNA or between two different regions of abnormality. In some embodiments, the LSTs are defined as the number of break points between regions longer than 10 Mb after filtering out regions shorter than 3 Mb. In certain embodiments, the HRD score is calculated as the sum of the LOH, TAI, and LST scores. Methods of determining an HRD score is available in the art, e.g., as described in Takaya et al., Sci Rep. 10(1):2757, 2020, Telli et al., Clin Cancer Res 22(15):3764-73, 2016, and Marchetti and McNeish, Cancer Breaking News 5(1):15-20, 2017. Further, commercial services for HRD score determination are also available, for example, services provided by Ambry Genetics, Caris Life Sciences, Counsylgenetic, Foundation Medicine, GeneDX, Integrated Genetics, Invitae, Myriad Genetics, and Neogenomics. In some embodiments, an individual having the genotype BRCA1wt, BRCA2 wt, or a combination thereof can be HRD-negative or HRD-positive. In other embodiments, a mutation in the BRCA1 and/or BRCA2 can lead to HRD. In other words, a mutation in BRCA1 and/or BRCA2 can lead to an individual having a HRD-positive status. In some embodiments, an individual having the genotype BRCA1wt, BRCA2 wt, and/or TP53m (e.g., BRCA1wt and BRCA2 wt, BRCA1wt and TP53m, BRCA2 wt and TP53m, and BRCA1wt and BRCA2 wt and TP53m) can be HRD-negative or HRD-positive. In some embodiments, an individual having the genotype BRCA1wt, BRCA2 wt, and/or TP53m (e.g., BRCA1wt and BRCA2 wt, BRCA1wt and TP53m, BRCA2 wt and TP53m, and BRCA1wt and BRCA2 wt and TP53m) is HRD-negative. In particular embodiments, an individual identified as having an HRD-positive status has an HRD score of 42 or greater (e.g., 42, 43, 44, 45, 46, 47, 48, 49, 50, 51, 52, 53, 54, 55, 56, 57, 58, 59, 60, or greater).

### Expression Vector

In some embodiments, the Granulocyte Macrophage Colony Stimulating Factor (GM-CSF) in the expression vector is a human GM-CSF sequence. In some embodiments, the expression vector further comprises a promoter, e.g., the promoter is a cytomegalovirus (CMV) mammalian promoter. In some embodiments, the mammalian CMV promoter comprises a CMV immediate early (IE) 5′ UTR enhancer sequence and a CMV IE Intron A. In further embodiments, the expression vector further comprises a CMV enhancer sequence and a CMV intron sequence.

The first insert and the second insert in the expression vector can be operably linked to the promoter. In particular embodiments, the expression vector further comprises a nucleic acid sequence encoding a picornaviral 2A ribosomal skip peptide between the first and the second nucleic acid inserts.

In some embodiments, the expression vector comprises at least one bifunctional shRNA (bi-shRNA). In some embodiments, the bi-shRNA comprises a first stem-loop structure that comprises an siRNA component and a second stem-loop structure that comprises a miRNA component. In some embodiments, the bi-functional shRNA has two mechanistic pathways of action, that of the siRNA and that of the miRNA. Thus, in some embodiments, the bi-functional shRNA described herein is different from a traditional shRNA, i.e., a DNA transcription derived RNA acting by the siRNA mechanism of action or from a “doublet shRNA” that refers to two shRNAs, each acting against the expression of two different genes but in the traditional siRNA mode. In some embodiments, the bi-shRNA incorporates siRNA (cleavage dependent) and miRNA (cleavage-independent) motifs.

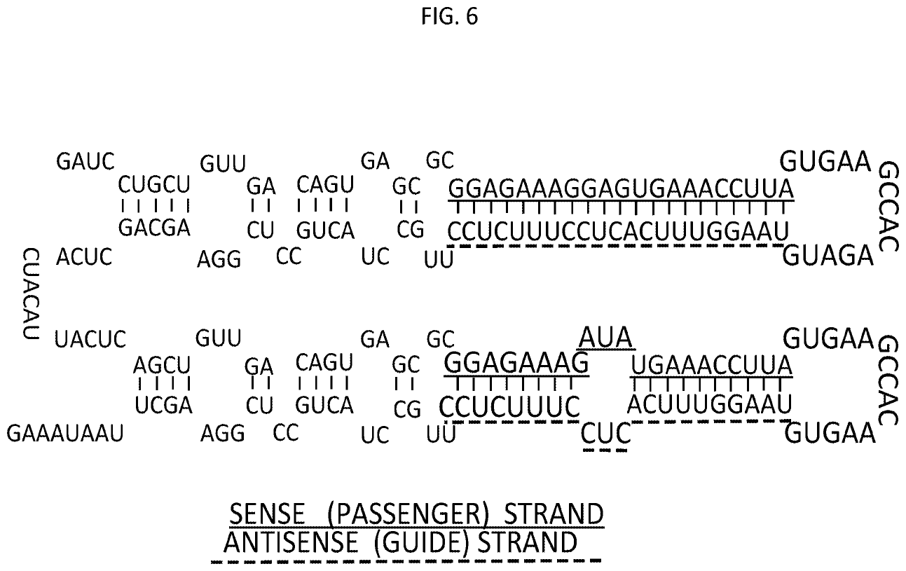

In some embodiments, the at least one bi-shRNA is capable of hybridizing to one of more regions of an mRNA transcript encoding furin. In some embodiments, the mRNA transcript encoding furin is a nucleic acid sequence of SEQ ID NO:1. In some embodiments, the one or more regions of the mRNA transcript encoding furin is selected from base sequences 300-318, 731-740, 1967-1991, 2425-2444, 2827-2851, and 2834-2852 of SEQ ID NO:1. In some embodiments, the expression vector targets the coding region of the furin mRNA transcript, the 3′ UTR region sequence of the furin mRNA transcript, or both the coding sequence and the 3′ UTR sequence of the furin mRNA transcript simultaneously. In some embodiments, the bi-shRNA comprises SEQ ID NO:2. In some embodiments, a bi-shRNA capable of hybridizing to one or more regions of an mRNA transcript encoding furin is referred to herein as bi-shRNA^{furin}. In some embodiments, the bi-shRNA^{furin} comprises or consists of two stem-loop structures each with miR-30a backbone. In some embodiments, a first stem-loop structure of the two stem-loop structures comprises complementary guiding strand and passenger strand (FIG. 6). In some embodiments, the second stem-loop structure of the two stem-loop structures comprises three mismatches in the passenger strand. In some embodiments, the three mismatches are at positions 9 to 11 in the passenger strand.

The expression vector can comprise: a. a first insert comprising a nucleic acid sequence encoding a Granulocyte Macrophage Colony Stimulating Factor (GM-CSF) sequence; and b. a second insert comprising two stem-loop structures each with a miR-30a loop; the first stem-loop structure has complete complementary guiding strand and passenger strand, while the second stem-loop structure has three base pair (bp) mismatches at positions 9 to 11 of the passenger strand. Descriptions of the miR-30a loop and its sequence are known in the art, see, e.g., Rao et al., Cancer Gene Ther. 17(11):780-91, 2010; Jay et al., Cancer Gene Ther. 20(12):683-9, 2013; Rao et al., Mol Ther. 24(8):1412-22, 2016; Phadke et al., DNA Cell Biol. 30(9):715-26, 2011; Barve et al., Mol Ther. 23(6):1123-1130, 2015; Rao et al., Methods Mol Biol. 942:259-78, 2013; and Senzer et al., Mol Ther. 20(3):679-86, 2012. In some embodiments, the miR-30a loop comprises the sequence of GUGAAGCCACAGAUG (SEQ ID NO:6). In some embodiments, the guiding strand in the first stem-loop structure comprises the sequence of SEQ ID NO:4 and the passenger strand in the first stem-loop structure has the sequence of SEQ ID NO:3. In some embodiments, the guiding strand in the second stem-loop structure comprises the sequence of SEQ ID NO:4 and the passenger strand in the second stem-loop structure has the sequence of SEQ ID NO: 5.

TABLE B
Sequences

| SEQ ID | | |
|---|---|---|
| NO | Description | Sequence |
| 1 | mRNA | GCGGGGAAGCAGCAGCGGCCAGGATGAATCCCAGGT |
| | transcript | GCTCTGGAGCTGGATGGTGAAGGTCGGCACTCTTCAC |
| | encoding | CCTCCCGAGCCCTGCCCGTCTCGGCCCCATGCCCCCAC |
| | furin | CAGTCAGCCCCGGGCCACAGGCAGTGAGCAGGCACCT |
| | | GGGAGCCGAGGCCCTGTGACCAGGCCAAGGAGACGG |
| | | GCGCTCCAGGGTCCCAGCCACCTGTCCCCCCCATGGA |
| | | GCTGAGGCCCTGGTTGCTATGGGTGGTAGCAGCAACA |
| | | GGAACCTTGGTCCTGCTAGCAGCTGATGCTCAGGGCC |
| | | AGAAGGTCTTCACCAACACGTGGGCTGTGCGCATCCC |
| | | TGGAGGCCCAGCGGTGGCCAACAGTGTGGCACGGAAG |
| | | CATGGGTTCCTCAACCTGGGCCAGATCTTCGGGGACT |
| | | ATTACCACTTCTGGCATCGAGGAGTGACGAAGCGGTC |
| | | CCTGTCGCCTCACCGCCCGCGGCACAGCCGGCTGCAG |
| | | AGGGAGCCTCAAGTACAGTGGCTGGAACAGCAGGTGG |
| | | CAAAGCGACGGACTAAACGGGACGTGTACCAGGAGC |
| | | CCACAGACCCCAAGTTTCCTCAGCAGTGGTACCTGTCT |
| | | GGTGTCACTCAGCGGGACCTGAATGTGAAGGCGGCCT |
| | | GGGCGCAGGGCTACACAGGGCACGGCATTGTGGTCTC |
| | | CATTCTGGACGATGGCATCGAGAAGAACCACCCGGAC |
| | | TTGGCAGGCAATTATGATCCTGGGGCCAGTTTTGATGT |
| | | CAATGACCAGGACCCTGACCCCCAGCCTCGGTACACA |
| | | CAGATGAATGACAACAGGCACGGCACACGGTGTGCGG |
| | | GGGAAGTGGCTGCGGTGGCCAACAACGGTGTCTGTGG |
| | | TGTAGGTGTGGCCTACAACGCCCGCATTGGAGGGGTG |
| | | CGCATGCTGGATGGCGAGGTGACAGATGCAGTGGAGG |
| | | CACGCTCGCTGGGCCTGAACCCCAACCACATCCACAT |
| | | CTACAGTGCCAGCTGGGGCCCCGAGGATGACGGCAAG |
| | | ACAGTGGATGGGCCAGCCCGCCTCGCCGAGGAGGCCT |
| | | TCTTCCGTGGGGTTAGCCAGGGCCGAGGGGGGCTGGG |
| | | CTCCATCTTTGTCTGGGCCTCGGGGAACGGGGGCCGG |
| | | GAACATGACAGCTGCAACTGCGACGGCTACACCAACA |
| | | GTATCTACACGCTGTCCATCAGCAGCGCCACGCAGTTT |
| | | GGCAACGTGCCGTGGTACAGCGAGGCCTGCTCGTCCA |
| | | CACTGGCCACGACCTACAGCAGTGGCAACCAGAATGA |
| | | GAAGCAGATCGTGACGACTGACTTGCGGCAGAAGTGC |
| | | ACGGAGTCTCACACGGGCACCTCAGCCTCTGCCCCCTT |
| | | AGCAGCCGGCATCATTGCTCTCACCCTGGAGGCCAAT |
| | | AAGAACCTCACATGGCGGGACATGCAACACCTGGTGG |
| | | TACAGACCTCGAAGCCAGCCCACCTCAATGCCAACGA |
| | | CTGGGCCACCAATGGTGTGGGCCGGAAAGTGAGCCAC |
| | | TCATATGGCTACGGGCTTTTGGACGCAGGCGCCATGG |
| | | TGGCCCTGGCCCAGAATTGGACCACAGTGGCCCCCCA |
| | | GCGGAAGTGCATCATCGACATCCTCACCGAGCCCAAA |
| | | GACATCGGGAAACGGCTCGAGGTGCGGAAGACCGTG |
| | | ACCGCGTGCCTGGGCGAGCCCAACCACATCACTCGGC |
| | | TGGAGCACGCTCAGGCGCGGCTCACCCTGTCCTATAA |
| | | TCGCCGTGGCGACCTGGCCATCCACCTGGTCAGCCCC |
| | | ATGGGCACCCGCTCCACCCTGCTGGCAGCCAGGCCAC |
| | | ATGACTACTCCGCAGATGGGTTTAATGACTGGGCCTTC |
| | | ATGACAACTCATTCCTGGGATGAGGATCCCTCTGGCG |
| | | AGTGGGTCCTAGAGATTGAAAACACCAGCGAAGCCAA |
| | | CAACTATGGGACGCTGACCAAGTTCACCCTCGTACTCT |
| | | ATGGCACCGCCCCTGAGGGGCTGCCCGTACCTCCAGA |
| | | AAGCAGTGGCTGCAAGACCCTCACGTCCAGTCAGGCC |
| | | TGTGTGGTGTGCGAGGAAGGCTTCTCCCTGCACCAGA |
| | | AGAGCTGTGTCCAGCACTGCCCTCCAGGGTTCGCCCCC |
| | | CAAGTCCTCGATACGCACTATAGCACCGAGAATGACG |
| | | TGGAGACCATCCGGGCCAGCGTCTGCGCCCCCTGCCA |
| | | CGCCTCATGTGCCACATGCCAGGGGCCGGCCCTGACA |
| | | GACTGCCTCAGCTGCCCCAGCCACGCCTCCTTGGACCC |
| | | TGTGGAGCAGACTTGCTCCCGGCAAAGCCAGAGCAGC |
| | | CGAGAGTCCCCGCCACAGCAGCAGCCACCTCGGCTGC |
| | | CCCCGGAGGTGGAGGCGGGGCAACGGCTGCGGGCAG |
| | | GGCTGCTGCCCTCACACCTGCCTGAGGTGGTGGCCGG |
| | | CCTCAGCTGCGCCTTCATCGTGCTGGTCTTCGTCACTG |
| | | TCTTCCTGGTCCTGCAGCTGCGCTCTGGCTTTAGTTTTC |
| | | GGGGGGTGAAGGTGTACACCATGGACCGTGGCCTCAT |
| | | CTCCTACAAGGGGCTGCCCCCTGAAGCCTGGCAGGAG |
| | | GAGTGCCCGTCTGACTCAGAAGAGGACGAGGGCCGGG |
| | | GCGAGAGGACCGCCTTTATCAAAGACCAGAGCGCCCT |
| | | CTGATGAGCCCACTGCCCACCCCCTCAAGCCAATCCCC |
| | | TCCTTGGGCACTTTTTAATTCACCAAAGTATTTTTTTAT |
| | | CTTGGGACTGGGTTTGGACCCCAGCTGGGAGGCAAGA |
| | | GGGGTGGAGACTGCTTCCCATCCTACCCTCGGGCCCA |
| | | CCTGGCCACCTGAGGTGGGCCCAGGACCAGCTGGGGC |
| | | GTGGGGAGGGCCGTACCCCACCCTCAGCACCCCTTCC |
| | | ATGTGGAGAAAGGAGTGAAACCTTTAGGGCAGCTTGC |
| | | CCCGGCCCCGGCCCCAGCCAGAGTTCCTGCGGAGTGA |
| | | AGAGGGGCAGCCCTTGCTTGTTGGGATTCCTGACCCA |
| | | GGCCGCAGCTCTTGCCCTTCCCTGTCCCTCTAAAGCAA |
| | | TAATGGTCCCATCCAGGCAGTCGGGGGCTGGCCTAGG |
| | | AGATATCTGAGGGAGGAGGCCACCTCTCCAAGGGCTT |
| | | CTGCACCCTCCACCCTGTCCCCCAGCTCTGGTGAGTCT |
| | | TGGCGGCAGCAGCCATCATAGGAAGGGACCAAGGCA |
| | | AGGCAGGTGCCTCCAGGTGTGCACGTGGCATGTGGCC |
| | | TGTGGCCTGTGTCCCATGACCCACCCCTGTGCTCCGTG |
| | | CCTCCACCACCACTGGCCACCAGGCTGGCGCAGCCAA |
| | | GGCCGAAGCTCTGGCTGAACCCTGTGCTGGTGTCCTG |
| | | ACCACCCTCCCCTCTCTTGCACCCGCCTCTCCCGTCAG |
| | | GGCCCAAGTCCCTGTTTTCTGAGCCCGGGCTGCCTGGG |
| | | CTGTTGGCACTCACAGACCTGGAGCCCCTGGGTGGGT |
| | | GGTGGGGAGGGGCGCTGGCCCAGCCGGCCTCTCTGGC |
| | | CTCCCACCCGATGCTGCTTTCCCCTGTGGGGATCTCAG |
| | | GGGCTGTTTGAGGATATATTTTCACTTTGTGATTATTT |
| | | CACTTTAGATGCTGATGATTTGTTTTTGTATTTTTAATG |
| | | GGGGTAGCAGCTGGACTACCCACGTTCTCACACCCAC |
| | | CGTCCGCCCTGCTCCTCCCTGGCTGCCCTGGCCCTGAG |
| | | GTGTGGGGGCTGCAGCATGTTGCTGAGGAGTGAGGAA |
| | | TAGTTGAGCCCCAAGTCCTGAAGAGGCGGGCCAGCCA |
| | | GGCGGGCTCAAGGAAAGGGGGTCCCAGTGGGAGGGG |
| | | CAGGCTGACATCTGTGTTTCAAGTGGGGCTCGCCATGC |
| | | CGGGGGTTCATAGGTCACTGGCTCTCCAAGTGCCAGA |
| | | GGTGGGCAGGTGGTGGCACTGAGCCCCCCCAACACTG |
| | | TGCCCTGGTGGAGAAAGCACTGACCTGTCATGCCCCC |
| | | CTCAAACCTCCTCTTCTGACGTGCCTTTTGCACCCCTC |
| | | CCATTAGGACAATCAGTCCCCTCCCATCTGGGAGTCCC |
| | | CTTTTCTTTTCTACCCTAGCCATTCCTGGTACCCAGCCA |
| | | TCTGCCCAGGGGTGCCCCCTCCTCTCCCATCCCCCTGC |
| | | CCTCGTGGCCAGCCCGGCTGGTTTTGTAAGATGCTGGG |
| | | TTGGTGCACAGTGATTTTTTTCTTGTAATTTAAACAGG |
| | | CCCAGCATTGCTGGTTCTATTTAATGGACATGAGATAA |
| | | TGTTAGAGGTTTTAAAGTGATTAAACGTGCAGACTAT |
| | | GCAAACCAG |
| 2 | bi-shRNA^{furin} | GAUCCUGCUGUUGACAGUGAGCGCGGAGAAAGGAGUGAA |
| | | ACCUUAGUGAAGCCACAGAUGUAAGGUUUCACUCCUUUC |
| | | UCCUUGCCUACUGCCUCGGAAGCAGCUCACUACAUUACUC |
| | | AGCUGUUGACAGUGAGCGCGGAGAAAGAUAUGAAACCUU |
| | | AGUGAAGCCACAGAUGUAAGGUUUCACUCCUUUCUCCUU |
| | | GCCUACUGCCUCGGAAGCUUAAUAAAGGAUCUUUUAUUU |
| | | UCAUUGGAUC |
| 3 | Passenger | GGAGAAAGGAGUGAAACCUUA |
| | strand in first | |
| | stem-loop | |
| | structure in bi- | |
| | shRNA^{furin} | |
| 4 | Guide strand | UAAGGUUUCACUCCUUUCUCC |
| | in both first | |
| | and second | |
| | stem-loop | |
| | structures in | |
| | bi-shRNA^{furin} | |
| 5 | Passenger | GGAGAAAGAUAUGAAACCUUA |
| | strand in | |
| | second stem- | |
| | loop structure | |
| | in bi- | |
| | shRNA^{furin} | |
| 6 | miR-30a loop | GUGAAGCCACAGAUG |

In some embodiments, the expression vector plasmid can have a sequence that is at least 90% (e.g., 90%, 91%, 92%, 93%, 94%, 95%, 96%, 97%, 98%, 99%, or 100%) identical to the sequence of SEQ ID NO:7. The vector plasmid can comprise a first nucleic acid insert operably linked to a promoter, wherein the first insert encodes the GM-CSF cDNA, a second nucleic acid insert operably linked to the promoter, wherein the second insert encodes one or more short hairpin RNAs (shRNA) capable of hybridizing to a region of a mRNA transcript encoding furin, thereby inhibiting furin expression via RNA interference.

SEQ ID NO: 7
GGCCATTGCATACGTTGTATCCATATCATAATATGTACATTTATATTGG
CTCATGTCCAACATTACCGCCATGTTGACATTGATTATTGACTAGTTAT
TAATAGTAATCAATTACGGGGTCATTAGTTCATAGCCCATATATGGAGT
TCCGCGTTACATAACTTACGGTAAATGGCCCGCCTGGCTGACCGCCCAA
CGACCCCCGCCCATTGACGTCAATAATGACGTATGTTCCCATAGTAACG
CCAATAGGGACTTTCCATTGACGTCAATGGGTGGAGTATTTACGGTAAA
CTGCCCACTTGGCAGTACATCAAGTGTATCATATGCCAAGTACGCCCCC
TATTGACGTCAATGACGGTAAATGGCCCGCCTGGCATTATGCCCAGTAC
ATGACCTTATGGGACTTTCCTACTTGGCAGTACATCTACGTATTAGTCA
TCGCTATTACCATGGTGATGCGGTTTTGGCAGTACATCAATGGGCGTGG
ATAGCGGTTTGACTCACGGGGATTTCCAAGTCTCCACCCCATTGACGTC
AATGGGAGTTTGTTTTGGCACCAAAATCAACGGGACTTTCCAAAATGTC
GTAACAACTCCGCCCCATTGACGCAAATGGGCGGTAGGCGTGTACGGTG
GGAGGTCTATATAAGCAGAGCTCGTTTAGTGAACCGTCAGATCGCCTGG
AGACGCCATCCACGCTGTTTTGACCTCCATAGAAGACACCGGGACCGAT
CCAGCCTCCGCGGCCGGGAACGGTGCATTGGAACGCGGATTCCCCGTGC
CAAGAGTGACGTAAGTACCGCCTATAGACTCTATAGGCACACCCCTTTG
GCTCTTATGCATGCTATACTGTTTTTGGCTTGGGGCCTATACACCCCCG
CTTCCTTATGCTATAGGTGATGGTATAGCTTAGCCTATAGGTGTGGGTT
ATTGACCATTATTGACCACTCCAACGGTGGAGGGCAGTGTAGTCTGAGC
AGTACTCGTTGCTGCCGCGCGCGCCACCAGACATAATAGCTGACAGACT
AACAGACTGTTCCTTTCCATGGGTCTTTTCTGCAGTCACCGTCGTCGAC
GGTATCGATAAGCTTGATATCGAATTCCGCTGGAGGATGTGGCTGCAGA
GCCTGCTGCTCTTGGGCACTGTGGCCTGCAGCATCTCTGCACCCGCCCG
CTCGCCCAGCCCCAGCACGCAGCCCTGGGAGCATGTGAATGCCATCCAG
GAGGCCCGGCGTCTCCTGAACCTGAGTAGAGACACTGCTGCTGAGATGA
ATGAAACAGTAGAAGTCATCTCAGAAATGTTTGACCTCCAGGAGCCGAC
CTGCCTACAGACCCGCCTGGAGCTGTACAAGCAGGGCCTGCGGGGCAGC
CTCACCAAGCTCAAGGGCCCCTTGACCATGATGGCCAGCCACTACAAGC
AGCACTGCCCTCCAACCCCGGAAACTTCCTGTGCAACCCAGACTATCAC
CTTTGAAAGTTTCAAAGAGAACCTGAAGGACTTTCTGCTTGTCATCCCC
TTTGACTGCTGGGAGCCAGTCCAGGAGTGAGACCGGCCAGATGAGGCTG
GCCAAGCCGGGGAGCTGCTCTCTCATGAAACAAGAGCTAGAAACTCAGG
ATGGTCATCTTGGAGGGACCAAGGGGTGGGCCACAGCCATGGTGGGAGT
GGCCTGGACCTGCCCTGGGCCACACTGACCCTGATACAGGCATGGCAGA
AGAATGGGAATATTTTATACTGACAGAAATCAGTAATATTTATATATTT
ATATTTTTAAAATATTTATTTATTTATTTATTTAAGTTCATATTCCATA
TTTATTCAAGATGTTTTACCGTAATAATTATTATTAAAAATATGCTTCT
AAAAAAAAAAAAAAAAAAAAAAACGGAATTCACGTGGGCCCGGTACCGT
ATACTCTAGAAGATCTGGCAGCGGAGAGGGCAGAGGAAGTCTTCTAACA
TGCGGTGACGTGGAGGAGAATCCCGGCCCTAGGATGTCTAGAGCGGCCG
CGGATCCTGCTGTTGACAGTGAGCGCGGAGAAAGGAGTGAAACCTTAGT
GAAGCCACAGATGTAAGGTTTCACTCCTTTCTCCTTGCCTACTGCCTCG
GAAGCAGCTCACTACATTACTCAGCTGTTGACAGTGAGCGCGGAGAAAG
ATATGAAACCTTAGTGAAGCCACAGATGTAAGGTTTCACTCCTTTCTCC
TTGCCTACTGCCTCGGAAGCTTAATAAAGGATCTTTTATTTTCATTGGA
TCCAGATCTTTTTCCCTCTGCCAAAAATTATGGGGACATCATGAAGCCC
CTTGAGCATCTGACTTCTGGCTAATAAAGGAAATTTATTTTCATTGCAA
TAGTGTGTTGGAATTTTTTGTGTCTCTCACTCGGAAGGACATATGGGAG
GGCAAATCATTTAAAACATCAGAATGAGTATTTGGTTTAGAGTTTGGCA
ACATATGCCCATTCTTCCGCTTCCTCGCTCACTGACTCGCTGCGCTCGG
TCGTTCGGCTGCGGCGAGCGGTATCAGCTCACTCAAAGGCGGTAATACG
GTTATCCACAGAATCAGGGGATAACGCAGGAAAGAACATGTGAGCAAAA
GGCCAGCAAAAGGCCAGGAACCGTAAAAAGGCCGCGTTGCTGGCGTTTT
TCCATAGGCTCCGCCCCCCTGACGAGCATCACAAAAATCGACGCTCAAG
TCAGAGGTGGCGAAACCCGACAGGACTATAAAGATACCAGGCGTTTCCC
CCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAG
CTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCG
GTAACTATCGTCTTGAGTCCAACCCGGTAAGACACGACTTATCGCCACT
GGCAGCAGCCACTGGTAACAGGATTAGCAGAGCGAGGTATGTAGGCGGT
GCTACAGAGTTCTTGAAGTGGTGGCCTAACTACGGCTACACTAGAAGAA
CAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAG
AAGATCCTTTGATCTTTTCTACGGGGTCTGACGCTCAGTGGAACGAAAA
CTCACGTTAAGGGATTTTGGTCATGAGATTATCAAAAAGGATCTTCACC
TAGATCCTTTTAAATTAAAAATGAAGTTTTAAATCAATCTAAAGTATAT
ATGAGTAAACTTGGTCTGACAGTTACCAATGCTTAATCAGTGAGGCACC
TATCTCAGCGATCTGTCTATTTCGTTCATCCATAGTTGCCTGACTCGGG
GGGGGGGGGCGCTGAGGTCTGCCTCGTGAAGAAGGTGTTGCTGACTCAT
ACCAGGCCTGAATCGCCCCATCATCCAGCCAGAAAGTGAGGGAGCCACG
GTTGATGAGAGCTTTGTTGTAGGTGGACCAGTTGGTGATTTTGAACTTT
TGCTTTGCCACGGAACGGTCTGCGTTGTCGGGAAGATGCGTGATCTGAT
CCTTCAACTCAGCAAAAGTTCGATTTATTCAACAAAGCCGCCGTCCCGT
CAAGTCAGCGTAATGCTCTGCCAGTGTTACAACCAATTAACCAATTCTG
ATTAGAAAAACTCATCGAGCATCAAATGAAACTGCAATTTATTCATATC
AGGATTATCAATACCATATTTTTGAAAAAGCCGTTTCTGTAATGAAGGA
GAAAACTCACCGAGGCAGTTCCATAGGATGGCAAGATCCTGGTATCGGT
CTGCGATTCCGACTCGTCCAACATCAATACAACCTATTAATTTCCCCTC
GTCAAAAATAAGGTTATCAAGTGAGAAATCACCATGAGTGACGACTGAA
TCCGGTGAGAATGGCAAAAGCTTATGCATTTCTTTCCAGACTTGTTCAA
CAGGCCAGCCATTACGCTCGTCATCAAAATCACTCGCATCAACCAAACC
GTTATTCATTCGTGATTGCGCCTGAGCGAGACGAAATACGCGATCGCTG
TTAAAAGGACAATTACAAACAGGAATCGAATGCAACCGGCGCAGGAACA
CTGCCAGCGCATCAACAATATTTTCACCTGAATCAGGATATTCTTCTAA
TACCTGGAATGCTGTTTTCCCGGGGATCGCAGTGGTGAGTAACCATGCA
TCATCAGGAGTACGGATAAAATGCTTGATGGTCGGAAGAGGCATAAATT
CCGTCAGCCAGTTTAGTCTGACCATCTCATCTGTAACATCATTGGCAAC
GCTACCTTTGCCATGTTTCAGAAACAACTCTGGCGCATCGGGCTTCCCA
TACAATCGATAGATTGTCGCACCTGATTGCCCGACATTATCGCGAGCCC
ATTTATACCCATATAAATCAGCATCCATGTTGGAATTTAATCGCGGCCT
CGAGCAAGACGTTTCCCGTTGAATATGGCTCATAACACCCCTTGTATTA
CTGTTTATGTAAGCAGACAGTTTTATTGTTCATGATGATATATTTTTAT
CTTGTGCAATGTAACATCAGAGATTTTGAGACACAACGTGGCTTTCCCC
CCCCCCCCATTATTGAAGCATTTATCAGGGTTATTGTCTCATGAGCGGA
TACATATTTGAATGTATTTAGAAAAATAAACAAATAGGGGTTCCGCGCA
CATTTCCCCGAAAAGTGCCACCTGACGTCTAAGAAACCATTATTATCAT
GACATTAACCTATAAAAATAGGCGTATCACGAGGCCCTTTCGTCTCGCG
CGTTTCGGTGATGACGGTGAAAACCTCTGACACATGCAGCTCCCGGAGA
CGGTCACAGCTTGTCTGTAAGCGGATGCCGGGAGCAGACAAGCCCGTCA
GGGCGCGTCAGCGGGTGTTGGCGGGTGTCGGGGCTGGCTTAACTATGCG
GCATCAGAGCAGATTGTACTGAGAGTGCACCATATGCGGTGTGAAATAC
CGCACAGATGCGTAAGGAGAAAATACCGCATCAGATTGGCTATT

An expression vector comprising a first nucleic acid encoding GM-CSF and a second nucleic acid encoding at least one bifunctional short hairpin RNA (bi-shRNA) capable of hybridizing to a region of an mRNA transcript encoding furin is referred to as a bishRNA^{furin}/GMCSF expression vector.

### Cancer Cells

In some embodiments, the expression vectors used in methods described herein are within autologous cancer cells, e.g., autologous tumor cells, xenograft expanded autologous tumor cells, allogeneic tumor cells, xenograft expanded allogeneic tumor cells, or combinations thereof. In some embodiments, the autologous cancer cell is transfected with the expression vector. In some embodiments, the cells are autologous tumor cells. In some embodiments, the allogenic tumor cells are established cell lines. In some embodiments, autologous tumor cells are obtained from the individual in need thereof. In some embodiments, when the cells are autologous tumor cells, the composition is referred to as an autologous tumor cell vaccine. In some embodiments, the autologous tumor cell vaccine comprises from 1×10⁶ cells to about 5×10⁷ cells, such as 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 1×10⁷ cells, 2×10⁷ cells, 3×10⁷ cells, 4×10⁷ cells, or 5×10⁷ cells.

In some embodiments, the cells are harvested from an individual. In some embodiments, the cells are harvested from a tissue of the individual. In some embodiments, the tissue is a tumor tissue. In some embodiments, the tumor tissue is ovarian tumor tissue. In some embodiments, the tumor tissue is harvested during a biopsy or a cytoreduction surgery on the individual. In some embodiments, the tumor tissue or cells from the tumor tissue are placed in an antibiotic solution in a sterile container. In some embodiments, the antibiotic solution comprises gentamicin, sodium chloride, or a combination thereof.

### Cancers and Survival

In some embodiments, the cancer is an HRD-negative, wild-type BRCA1/2 cancer. In some embodiments, the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer. Examples of solid tumor cancers include, but are not limited to, endometrial cancer, biliary cancer, bladder cancer, liver hepatocellular carcinoma, gastric/esophageal cancer, ovarian cancer, melanoma, breast cancer, pancreatic cancer, colorectal cancer, glioma, non-small-cell lung carcinoma, prostate cancer, cervical cancer, kidney cancer, thyroid cancer, a neuroendocrine cancer, small cell lung cancer, a sarcoma, head and neck cancer, brain cancer, clear cell renal cell carcinoma, skin cancer, endocrine tumor, thyroid cancer, tumor of unknown origin, and a gastrointestinal stromal tumor.

In particular embodiments of the methods, the cancer is ovarian cancer. In some embodiments, the method can prevent or delay relapse of a substantially eradicated ovarian cancer. The substantially eradicated ovarian cancer can be Stage III or Stage IV ovarian cancer. In other embodiments, the cancer can be breast cancer, melanoma, or lung cancer. In some embodiments, Stage III ovarian cancer means that the cancer is found in one or both ovaries and has spread outside the pelvis to other parts of the abdomen and/or nearby lymph nodes. It is also considered Stage III ovarian cancer when it has spread to the surface of the liver. In Stage IV ovarian cancer, the cancer has spread beyond the abdomen to other parts of the body, such as the lungs or tissue inside the liver. Cancer cells in the fluid around the lungs is also considered Stage IV ovarian cancer.

In certain embodiments, the ovarian cancer is Stage III or Stage IV ovarian cancer. In some embodiments, the Stage III ovarian cancer is Stage IIIb or worse. In some embodiments, the ovarian cancer is a high-grade serous ovarian carcinoma, a clear cell ovarian carcinoma, endometroid ovarian carcinoma, mucinous ovarian carcinoma, or a low-grade serous ovarian carcinoma.

In some embodiments of the methods, a relapse free survival (RFS) of the individual is increased relative to an individual with substantially eradicated ovarian cancer who has not been administered the expression vector or autologous tumor cell vaccine containing the expression vector.

As used herein, the term “relapse free survival” refers to the time after administration of an initial therapy to treat a cancer that the cancer remains undetectable (i.e., until the cancer relapses). In some embodiments, relapse free survival of an individual receiving the expression vector or the autologous cancer cell vaccine containing the expression vector is from 5 months to 11 months longer than relapse free survival of an individual not receiving the expression vector or the autologous cancer cell vaccine containing the expression vector. In some embodiments, relapse free survival of an individual receiving the expression vector or the autologous cancer cell vaccine containing the expression vector is at least 5 months, 6 months, 7 months 8 months, 9 months, 10 months, or 11 months longer than relapse free survival of an individual not receiving the expression vector or the autologous cancer cell vaccine containing the expression vector.

As used herein, the term “substantially eradicated” refers to an ovarian cancer which is not detectable in an individual following an initial therapy to treat the ovarian cancer. In some embodiments, detection of ovarian cancer, or lack thereof, is by a chest x-ray, computed tomography (CT) scan, magnetic resonance imaging (MRI), detection of a cancer antigen 125 (CA-125) level, physical examination or presence of symptoms suggestive of active cancer, or any combination thereof. In some embodiments, a detection of cancer antigen 125 (CA-125) levels of ≤35 units/ml indicates no ovarian cancer is present in the individual. In some embodiments, an ovarian cancer which has been substantially eradicated can be referred to as having achieved a clinical complete response (cCR).

In some embodiments, relapse free survival of an individual receiving the expression vector or the autologous cancer cell vaccine containing the expression vector is at least 5 months longer than relapse free survival of an individual not receiving the expression vector or the autologous cancer cell vaccine containing the expression vector. In some embodiments, relapse free survival of a BRCAwt individual receiving the expression vector or the autologous cancer cell vaccine containing the expression vector is greater than 15 months from time of surgical debulking, wherein a relapse free survival of an individual not receiving the expression vector or the autologous cancer cell vaccine containing the expression vector is less than 15 months from time of surgical debulking. In some embodiments, relapse free survival of a BRCAwt individual receiving the expression vector or the autologous cancer cell vaccine containing the expression vector is at least 11 months longer than relapse free survival of an individual not receiving the expression vector or the autologous cancer cell vaccine containing the expression vector.

In some embodiments, the individual received an initial therapy. In some embodiments, administration of an initial therapy results in a clinical completely response of the cancer to the therapy. In some embodiments, the initial therapy comprises debulking, administration of a chemotherapy, administration of a therapeutic agent, or the combination thereof. In some embodiments, the chemotherapy comprises a platinum-based drug, a taxane, or a combination thereof. In some embodiments, the platinum-based drug comprises cisplatin, carboplatin, oxaliplatin, nedaplatin, triplatin tetranitrate, phenanthriplatin, picoplatin, satraplatin, or a combination thereof. In some embodiments, the platinum-based drug comprises carboplatin. In some embodiments, the taxane comprises paclitaxel, docetaxel, cabazitaxel, or a combination thereof. In some embodiments, the taxane comprises paclitaxel. In some embodiments, the therapeutic agent comprises an angiogenesis inhibitor, a PARP inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the PARP inhibitor comprises olaparib, rucaparib, niraparib, talazoparib, veliparib, pamiparib, or a combination thereof. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof. In some embodiments, the ovarian cancer is resistant or refractory to the chemotherapy or the therapeutic agent.

### Administration, Formulations, and Dosing

In some embodiments, the autologous cancer cell vaccine containing the expression vector comprises about 1×10⁶ or about 1×10⁷ autologous cancer cells transfected as described herein. In some embodiments, the autologous cancer cell vaccine comprises at least 1×10⁶ or at least 1×10⁷ autologous cancer cells transfected as described herein. In some embodiments, the autologous cancer cell vaccine comprises from about 1×10⁶ cells to about 1×10⁷ (e.g., 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, or 1×10⁷) autologous cancer cells transfected as described herein. In some embodiments, the autologous cancer cell vaccine comprises from about 1×10⁶ cells to about 2.5×10⁷ (e.g., 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, or 2.5×10⁷) autologous cancer cells transfected as described herein. In some embodiments, the autologous cancer cell vaccine comprises from about 1×10⁶ cells to about 5×10⁷ (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, or 5×10⁷) autologous cancer cells transfected as described herein.

In some embodiments, the autologous cancer cell vaccine further comprises one or more vaccine adjuvants.

In some embodiments, the expression vector or the autologous cancer cell vaccine is in a unit dosage form. The term “unit dosage form”, as used herein, describes a physically discrete unit containing a predetermined quantity of the expression vector or the autologous cancer cell vaccine described herein, in association with other ingredients (e.g., vaccine adjuvants). In some embodiments, the predetermined quantity is a number of cells.

In some embodiments, an individual is administered one dose of the expression vector or the autologous cancer cell vaccine per month. In some embodiments, a dose of the expression vector or the autologous cancer cell vaccine is administered to the individual once a month for from 1 months to 12 months. In some embodiments, the individual is administered at least one dose of the expression vector or the autologous cancer cell vaccine. In some embodiments, the individual is administered no more than twelve doses of the expression vector or the autologous cancer cell vaccine. In some embodiments, the individual is administered 1, 2, 3, 4, 5, 6, 7, 8, 9, 10, 11, or 12 doses of the expression vector or the autologous cancer cell vaccine. In some embodiments, the dose is a unit dosage form of the expression vector or the autologous cancer cell vaccine. In some embodiments, a dose of the expression vector or the autologous cancer cell vaccine is administered to the individual every three months, every two months, once a month, twice a month, or three times a month. In some embodiments, the expression vector or the autologous cancer cell vaccine is administered to the individual for up to 1 month, 2 months, 3 months, 4 months, 5 months, 6 months, 12 months, 18 months, 24 months, or 36 months. In some embodiments, the expression vector or the autologous cancer cell vaccine is administered to the individual by injection. In some embodiments, the injection is an intradermal injection. In some embodiments, a first dose of the expression vector or the autologous cancer cell vaccine is administered to the individual following confirmation of the individual achieving a clinical complete response (cCR). In some embodiments, a first dose of the expression vector or the autologous cancer cell vaccine is administered to the individual no earlier than the same day as the final treatment of the initial therapy. In some embodiments, a first dose of the expression vector or the autologous cancer cell vaccine is administered to the individual no later than 8 weeks following the final treatment of the initial therapy.

### Combinations

In some embodiments, the expression vector or the autologous cancer cell vaccine is administered with an additional therapeutic agent. In some embodiments, the additional therapeutic agent comprises a therapeutically effective dose of γIFN (gamma interferon). In some embodiments, the therapeutically effective dose of γIFN is from about 50 μg/m² to about 100 g/m². In some embodiments, the therapeutically effective dose of γIFN is about 50 μg/m², about 60 μg/m², about 70 μg/m², about 80 μg/m², about 90 μg/m², or about 100 μg/m². In some embodiments, the additional therapeutic agent comprises an angiogenesis inhibitor, a PARP inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the PARP inhibitor comprises olaparib, rucaparib, niraparib, talazoparib, veliparib, pamiparib, or a combination thereof. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof.

### EXAMPLES

### Example 1—Network Based Analysis of Genomic Variant Data Identifies TP53m-BRCA1/2 wt Homologous Recombination Proficient (HRP) Population as Most Sensitive to Vigil® Immunotherapy

This experiment describes further molecular analysis of genomic variant data in patients receiving Vigil® or placebo in a randomized double-blind trial to treat frontline Stage III/IV ovarian cancer. We sought to objectively and independently identify significant genomic variants, meaningful variant combinations, and relevant genes at the intersection or “hub” of ovarian cancer pathways.

### Methods

### Data Management and Design

Tumor annotated DNA polymorphism data was generated across 981 validated genes for all patients who entered into the phase IIb double blind randomized placebo controlled trial comparing Vigil® and Placebo for stage III/IV resectable ovarian cancer. Patient demographics, trial design and vaccine manufacturing were previously described [31]. Rocconi et al. demonstrated improved clinical benefit from randomization (done just prior to maintenance therapy initiation) in RFS (HR 0.51; CI 90% 0.30-0.88; p=0.02) and OS (HR 0.49; 90% CI 0.24-1.0 p=0.049) with Vigil® treatment in BRCAwt patients [31]. DNA samples of malignant tissue were analyzed from all 91 patients entered into trial and results were compared to clinical endpoints prospectively identified in the study statistical plan. Gene variants were classified as benign, likely benign, likely pathogenic, pathogenic, or uncertain significance per the ORB algorithm, which searches current literature across a multitude of databases.

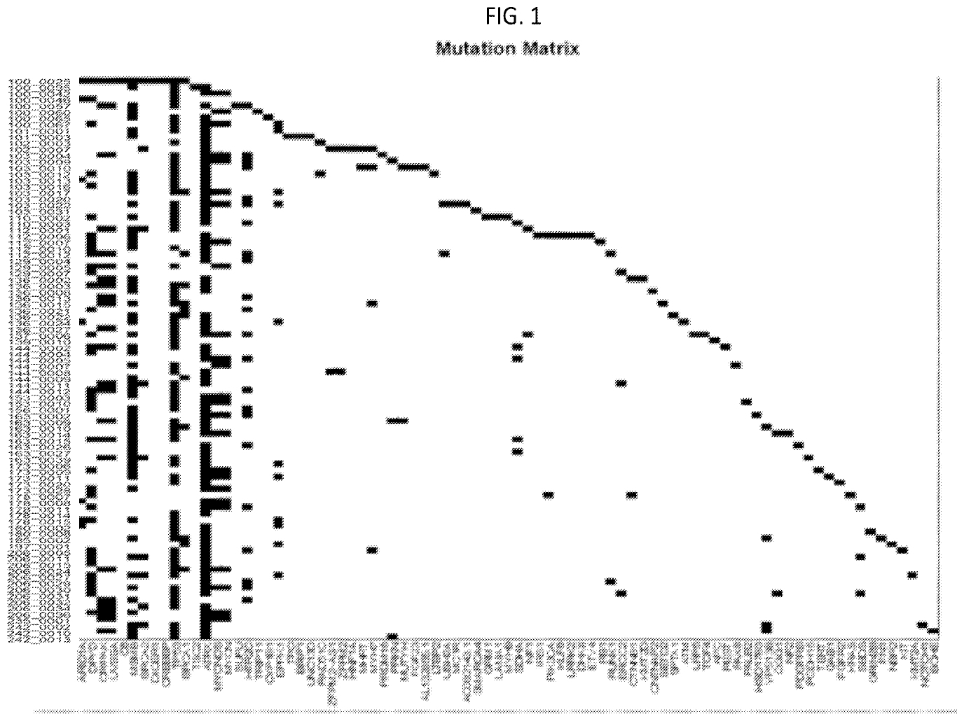

We considered only gene variants which were determined to be pathogenic or likely pathogenic, and these will be referred to as pathogenic mutations. Individual gene sets of pathogenically mutated genes for each patient in the trial were generated. An overall gene set was then constructed by taking the unique union (i.e., combining all of the individual gene sets and removing duplicated genes) of the individual gene sets. A binary mutation matrix is constructed from this overall gene set such that element (i,j) of the mutation matrix is equal to 1 if patient i has a pathogenic mutation in gene j and equal to 0 if patient i is wild type in gene j. Formatting the data in this way allows for easy identification of the mutation status of patients. Visual display of this mutation matrix of all patients is shown in FIG. 1. The mutation matrix allows for visualization of common mutations and genetic profiles, but does not explore the functional relationship between genes.

From the 83 pathogenic gene list, 77 genes were recognized and analyzed using the STRING application in Cytoscape to gauge functional interaction. To analyze how these pathogenically mutated genes functionally interact with each other, we constructed and explored properties via a STRING constructed protein-protein interaction (PPI) network. In the STRING network, an association or interaction may refer to direct (e.g., physical binding) or indirect interactions, such as shared participation in a common metabolic pathway [32]. Once the network was constructed, a topological distance between gene pairs was calculated, which allows us to define critical properties of the network. We further explored RFS and OS relationship of patients with varying mutational statuses in (i) hub genes, (ii) gene pairs with small topological distances and (iii) gene pairs with high cumulative C-scores. Finally, we stratified patients by tumor mutational burden high versus low to examine and compare RFS and OS difference.

### Tumor Mutation Burden (TMB) Analysis

The tumor annotated DNA polymorphism data generated across all 981 validated genes was utilized to determine the TMB (ORB) for every patient. TMB was calculated using synonymous and non-synonymous mutations as well as insertions and deletions (indels) per megabase of tumor genome. Patients were divided into high TMB (≥10) or low (<10) TMB and merged with clinical data from each patient. The difference in OS and RFS was calculated between the two group using Kaplan Meier (KM) analysis.

### Topological Distance

The overall gene list was inputted into the STRING application in Cytoscape [33]. From the total 83 genes, 77 were recognized by the STRING application and used to generate the PPI network. The application accesses the STRING database and generates a network for the input genes which consists of genes and their interactions, represented by vertices and edges, respectively. Genes are only connected via edges in the network if there is evidence they interact from published literature and high throughput experimental data. STRING uses this information to assign confidence scores, which we denote as s(i,j) to each interaction or edge. Individual STRING scores are produced for each of the interaction types and these scores are integrated to give a combined confidence score, s(i,j), between each pair of proteins. Each protein-protein interaction (PPI) score is bound between 0 and 1 which indicates how likely STRING judges the particular interaction to be true, given available evidence [1]. Next, edge weights between each pair of genes are calculated according to the following formula: w(i, j)=10(1−s(i, j)).

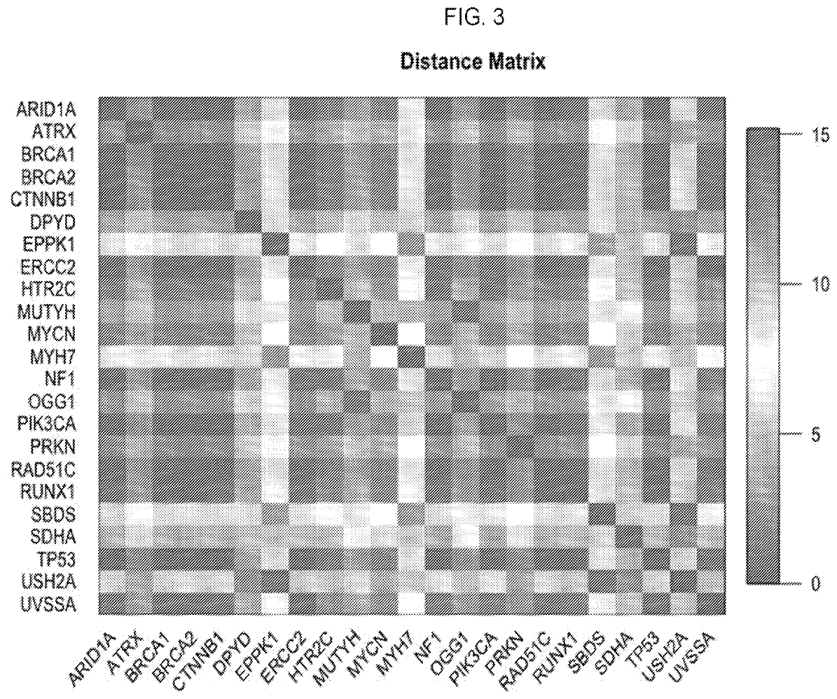

We chose to subtract the score, s(i,j) from one so that intuitively a small weight corresponds to strong evidence of a biological interaction between a gene pair. Additionally, this quantity is multiplied by 10 to shift the values to the desired scale [34]. Dijkstra's Algorithm is then used to calculate the length of the shortest weighted path between genes denoted, d(i,j). This is done by summing over the weighted edges that connect them and systematically finding the shortest path. From this, we arrived at the Distance Matrix seen in FIG. 3. For our purposes we took genes with distance less than or equal to 3.8, d(i,j)≤3.8, which is the bottom quarter. Intuitively, when a gene pair has a low topological distance, d(i,j), we are confident that these genes interact biologically. This inverse relationship between distance and level of interaction is critical to determine whether gene mutation pairs contribute to patient sensitivity and increased response to Vigil®.

### Analysis of STRING Network for Hub Genes

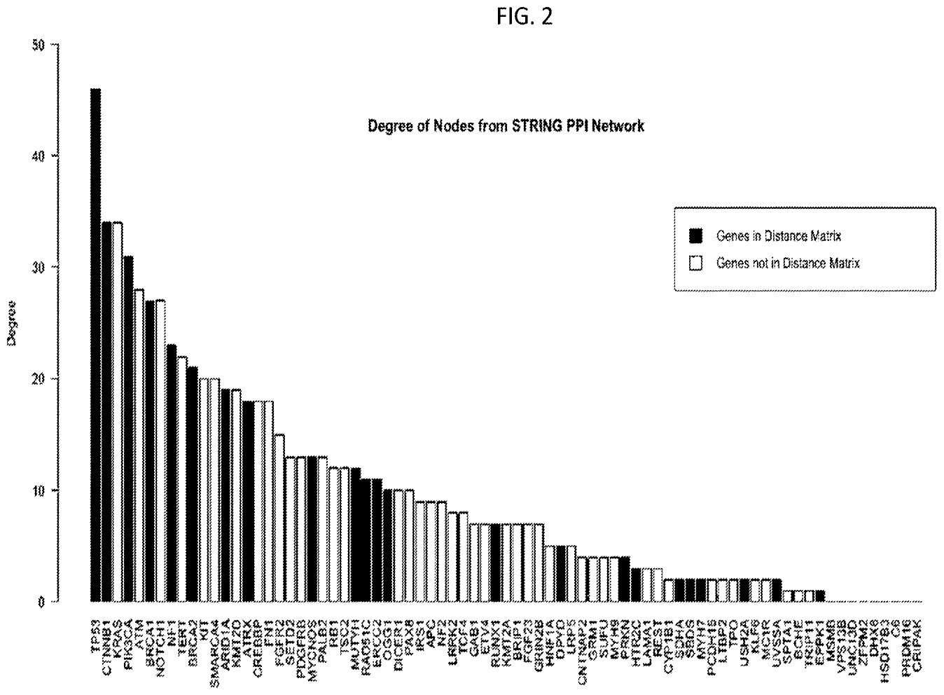

The STRING database has been maintained since 2000 (see, e.g., Szklarczyk et al., Nucleic Acids Res. 45(Database issue):D362-D368, 2017). A hub gene is defined to be a gene with a high degree of associations to other genes in the network. The degree of a gene or node is the number of connections it has to other genes or nodes in the network. Here, we considered a hub gene to be a gene with degree greater than or equal to 12 associations. A threshold of 12 selects the top quartile of genes based on the mean degree of genes in the network. A bar chart displaying the degrees of each gene in the overall set is shown in FIG. 2.

### C-Scores

The independent concepts of patient mutation profiles and the STRING Network are integrated by a C-score. We began by calculating the probability of co-mutation for every pair of genes in our overall gene set. The probability of a co-mutation is defined as the total number of the 91 patients who have a mutation in both of the genes in the given gene pair divided by a measure of the total number of times both genes are mutated individually, given by:

$$P ( i , j ) = \frac{\vert G ( i ) ∩ G ( j ) \vert}{\sqrt{m ( i ) * m ( j )}}$$

[34]. Where |G(i)∩G(j)| represents the number of individual tumors where both genes i and j are mutated, and m(i) and m(j) are the cumulative mutations of genes i and j, respectively. The range of P(i,j) is between 0 and 1 where P(i,j)=0 indicates that genes i and j never co-mutate and P(i, j)=1 means the genes will always co-mutate.

The probability of co-mutation and the topological distance between genes in the STRING network were then combined to calculate a C-score, denoted C(i,j), to quantify the likelihood that the genes interact functionally, termed “putative genetic interactions” [34]. The C-score is calculated by dividing the probability of co-mutation by the topological distance from the STRING network squared.

$$C ( i , j ) = \frac{P ( i , j )}{{d ( i , j )}^{2}} \frac{\vert G ( i ) ∩ G ( j ) \vert}{\sqrt{m ( i ) * m ( j )} {d ( i , j )}^{2}}$$

Further, the cumulative c-score for a gene i is denoted, cumC(i)=Σ_{i≠j}C(i, j). A gene with a high cumulative C-score is more likely to co-mutate with genes close to it in the STRING Network. The Individualized Network-based Co-Mutation (INCM) C-score is conceptually a measure of putative genetic interaction, or whether two genes can be assumed to interact. Liu et al. defined INCM and found that cumC-score is associated with increased response/sensitivity to various treatments in their original study [35].

### Results

### TMB Analysis

TMB for each patient was estimated using the mutational profile generated from the 981 gene panel using the combination of synonymous and non-synonymous mutations as well as indels. Patients were stratified into two groups; high TMB (≥10) or low (<10) TMB. In the Vigil® group, 33 patients had high TMB and 14 low TMB, similar results were observed in the placebo group of 34 and 10, respectively. No statistical difference in RFS or OS was observed in patients with high or low TMB receiving Vigil®1? compared to placebo from time of randomization (HR=0.60 90% CI 0.35-1.01; p=0.052 and HR=1.01 90% CI 0.41-2.52; p=0.49) (data not shown). TMB high did not alter significance of BRCAwt correlation with RFS previously reported [28]. BRCAwt, high TMB patients demonstrated improved RFS (HR=0.43 90% CI 0.23-0.78; p=0.009) and OS (HR=0.42 90% CI 0.19 to 0.93; p=0.032) from randomization. RFS and OS could not be evaluated in the BRCAwt, low TMB group due to small sample size.

### Network Construction and Pathway Enrichment Analysis

The STRING-constructed PPI network was made. As noted in methods, 83 of 981 genes analyzed were defined as pathogenic. Analysis was conducted by inputting each gene in the STRING network into the WikiPathways Application in Cytoscape [33]. For each gene, we extracted a list of pathways for which the gene is involved in. We then stratified the generated list of pathways into the six following categories: DNA repair, chromosomal organization and transcription, regulation of translational and post-translational modification, immunity, other cancer genes and not defined. Genes in the “not defined” category did not fit in one of the five selected pathways and/or did not have any known pathways in WikiPathways. Each gene in the STRING network was attributed to one or several of the above pathways, and pathways were sorted by the above categories.

### Single Gene Analysis

Hub Genes. Ten of the 77 genes in the PPI network were identified to have a degree greater than 12 associations, making them hub genes: TP53, CTNNB1, PIK3CA, BRCA1, NF1, BRCA2, ARID1A, ATRX, MYCNOS, MUTYH (FIG. 2).

We stratified by hub gene mutation status (mutant or wild type) and treatment group to perform KM analysis and log rank tests to assess if Vigil® demonstrated an advantage over placebo (p=≤0.1). RFS from randomization was the primary endpoint of the VITAL trial, therefore it was used as the primary benefit of this analysis. TP53, BRCA1 and BRCA2 were the only hub genes that reached our statistical cut off of p=≤0.1 from randomization. In patients with TP53m tumors, median RFS was 18.69 months with Vigil® and 8.35 months with placebo (one-sided p=0.096). In the BRCA1wt population, RFS was 12.75 months for Vigil® and 8.38 months for placebo (one-sided p=0.10). RFS for the BRCA2 wt population was 11.47 months and 8.35 months for Vigil® and placebo, respectively (one-sided p=0.05).

### Gene Pair Analysis

Gene Pairs with a Small Topological Distance. The maximum and minimum distance in the matrix are between genes USH2A and EPPK1, with a distance of 15.19 and between genes BRCA1 and BRCA2 with a distance of 0.02, respectively. We further analyzed gene pairs with a small topological distance. Using a cutoff of 3.8 which was determined by taking the range, 15.19 minus 0.02, and dividing by four, denoted the bottom quartile, we arrived at 139 gene pairs (FIG. 3).

We find that TP53 and BRCA1 have a distance of 0.04 and TP53 and BRCA2 have a distance of 0.06. This indicates that there is strong evidence that TP53 has a functional association with BRCA1 and BRCA2 and thus considered both BRCA1 and BRCA2 as a joint relationship designated as BRCA which is consistent with prior analysis of others.

Gene Pairs with High Cumulative C-Scores. After computing the C-Score for all gene pairs in the Distance Matrix (FIG. 3) and ordering genes from highest to lowest cumulative C-Score, cumC, the genes with highest cumC in order are BRCA1, BRCA2 and TP53. This indicates high connectivity within the network both from a co-mutation standpoint and a topological distance perspective. Thus, the function or dysfunction of these 3 genes has broad-reaching consequences for the other proteins within the ovarian cancer cell, more so than other genomic variants. This warranted closer attention to the effects of wild type versus mutant expression of BRCA and TP53.

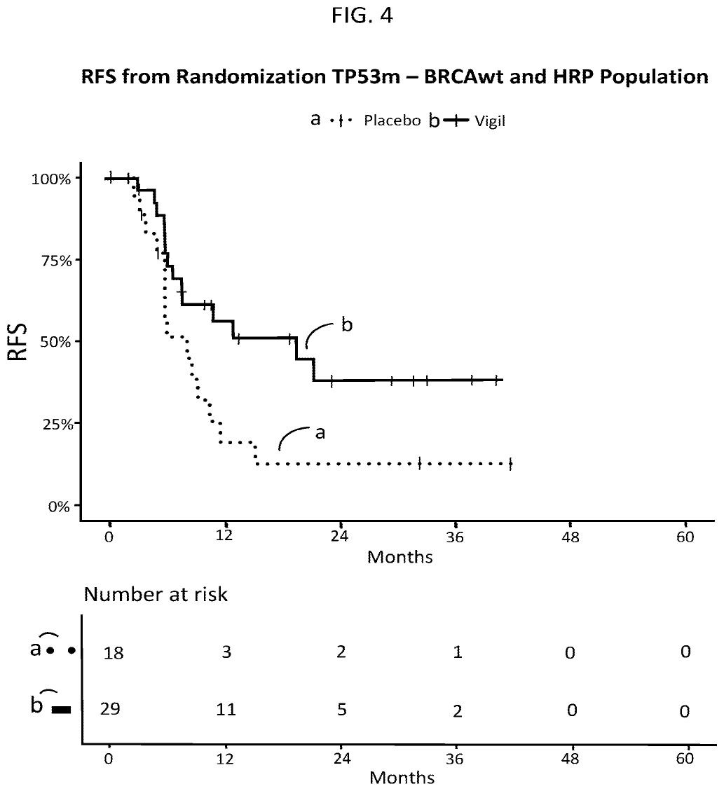

Given the proximity of TP53 with BRCA1 and BRCA2 in the STRING network and their high cumulative C-scores, we performed survival analysis across the four mutation statuses (co-mutant, mutant-wild type, wild type-mutant and co-wild type). Impact of these combinations on relapse free survival in the Vigil® treatment group compared to placebo is displayed in Tables 1-3. The TP53m-BRCAwt group experienced a median RFS of 19.35 months, compared to 11.71 months in dual-mutant and 10.48 months in dual-wild type. Compared between Vigil® and placebo arms, the TP53m-BRCAwt group had median RFS of 19.35 months compared to 7.85 months (p=0.01, HR=0.44) (FIG. 4). Additional tests demonstrated statistical significance (p<0.05) for the combination of BRCA and our identified hub genes, displayed in Table 4. Results regarding other genes and gene pairs with small distances are presented in Tables 5-7.

TABLE 1
RFS from randomization for TP53 and BRCA

| Gene 1 | Gene 2 | Vigil ® Median | Placebo Median | Difference | nVigil ® | nPlacebo | PVAL | HR |
|---|---|---|---|---|---|---|---|---|
| BRCAm | TP53m | 11.71 | 14.75 | −3.04 | 4 | 14 | 0.27 | 1.50 |
| BRCAwt | TP53m | 19.35 | 7.85 | 11.50 | 29 | 18 | 0.013 | 0.44 |
| BRCAm | TP53wt | 10.48 | 31.90 | −21.42 | 3 | 3 | 0.39 | 1.40 |
| BRCAwt | TP53wt | 10.48 | 8.38 | 2.10 | 11 | 9 | 0.47 | 1.05 |

TABLE 2
RFS from randomization for TP53 with BRCA1 and BRCA2 individually

| | | Vigil ® | Placebo | | | | |
|---|---|---|---|---|---|---|---|
| Gene 1 | Gene 2 | Median | Median | Difference | nVigil | nPlacebo | PVAL |
| TP53m | BRCA1m | 4.731 | 14.7515 | −10.0205 | 3 | 5 | 0.252 |
| TP53m | BRCA1wt | 19.3511 | 8.345 | 11.0062 | 30 | 27 | 0.0515 |
| TP53wt | BRCA1wt | 10.4805 | 13.9959 | −3.5154 | 12 | 10 | 0.3932 |
| TP53m | BRCA2wt | 12.7474 | 8.0493 | 4.6982 | 32 | 25 | 0.0335 |
| TP53wt | BRCA2wt | 10.4805 | 8.3778 | 2.1027 | 14 | 11 | 0.4086 |

*TP53wt-BRCA1m was unable to be performed due to small sample size
*TP53m-BRCA2m and TP53wt-BRCA2m was unable to be performed due to small sample size

TABLE 3
RFS from procurement for TP53 with BRCA1 and BRCA2 individually

| | | Vigil ® | Placebo | | | | |
|---|---|---|---|---|---|---|---|
| Gene 1 | Gene 2 | Median | Median | Difference | nVigil | nPlacebo | PVAL |
| TP53m | BRCA1m | 17.577 | 20.271 | −2.694 | 3 | 5 | 0.3499 |
| TP53m | BRCA1wt | 26.9076 | 15.8686 | 11.039 | 30 | 27 | 0.0478 |
| TP53wt | BRCA1wt | 18.2998 | 20.2546 | −1.9548 | 12 | 10 | 0.4551 |
| TP53m | BRCA2wt | 19.3511 | 15.8686 | 3.4825 | 32 | 25 | 0.0244 |
| TP53wt | BRCA2wt | 18.2998 | 14.9158 | 3.384 | 14 | 11 | 0.3578 |

*TP53wt-BRCA1m was unable to be performed due to small sample size
*TP53m-BRCA2m and TP53wt-BRCA2m was unable to be performed due to small sample size

TABLE 4
RFS for BRCAwt and other hub genes of significance

| | | Vigil ® | Placebo | | | | | |
|---|---|---|---|---|---|---|---|---|
| Gene 1 | Gene 2 | Median | Median | Difference | nVigil ® | nPlacebo | PVAL | HR |
| BRCAwt | PIK3CAwt | 11.47 | 7.95 | 3.52 | 39 | 26 | 0.02 | 0.53 |
| BRCAwt | NF1wt | 12.75 | 8.35 | 4.40 | 40 | 26 | 0.04 | 0.59 |
| BRCAwt | ARID1Awt | 12.75 | 7.95 | 4.80 | 37 | 24 | 0.05 | 0.59 |
| BRCAwt | MYCNOSwt | 13.67 | 5.72 | 7.95 | 27 | 17 | 0.03 | 0.47 |
| BRCAwt | MUTYHwt | 12.75 | 7.95 | 4.80 | 38 | 27 | 0.03 | 0.56 |

TABLE 5
RFS from randomization - small distance gene pairs

| | | Vigil ® | Placebo | | | | |
|---|---|---|---|---|---|---|---|
| Gene 1 | Gene 2 | Median | Median | Difference | nVigil | nPlacebo | PVAL |
| UVSSAm | ATRXm | NR | 11.7947 | NR | 9 | 6 | 0.2556 |
| UVSSAwt | ATRXm | 11.4661 | 8.3778 | 3.0883 | 32 | 28 | 0.359 |
| UVSSAwt | ATRXwt | NR | 8.4928 | NR | 4 | 4 | 0.0386 |
| BRCA2wt | BRCA1m | 4.731 | 11.4004 | −6.6694 | 5 | 6 | 0.4507 |
| BRCA2wt | BRCA1wt | 11.4661 | 8.345 | 3.1211 | 41 | 30 | 0.0354 |
| MUTYHwt | BRCA2wt | 11.4661 | 8.345 | 3.1211 | 44 | 36 | 0.0458 |
| PIK3CAwt | BRCA2wt | 10.6448 | 8.0493 | 2.5955 | 45 | 35 | 0.0351 |
| PRKNwt | BRCA2wt | 11.4661 | 8.0493 | 3.4168 | 44 | 35 | 0.0357 |
| RAD51Cwt | BRCA2wt | 11.4661 | 8.345 | 3.1211 | 45 | 35 | 0.0464 |
| RUNX1wt | BRCA2wt | 11.4661 | 8.0493 | 3.4168 | 45 | 35 | 0.0379 |
| TP53m | ERCC2wt | 18.694 | 8.345 | 10.3491 | 33 | 30 | 0.0577 |
| TP53wt | ERCC2wt | 10.4805 | 19.614 | −9.1335 | 14 | 11 | 0.432 |
| TP53m | HTR2Cm | 12.7474 | 5.7823 | 6.9651 | 10 | 3 | 0.0395 |
| TP53m | HTR2Cwt | 18.694 | 8.9692 | 9.7248 | 23 | 29 | 0.213 |
| TP53wt | HTR2Cwt | 13.6674 | 8.1643 | 5.5031 | 7 | 10 | 0.3037 |
| TP53m | MYCNm | NR | 8.5092 | NR | 7 | 8 | 0.0592 |
| TP53m | MYCNwt | 18.694 | 8.345 | 10.3491 | 26 | 24 | 0.3284 |
| TP53wt | MYCNm | 5.7823 | 13.9959 | −8.2136 | 6 | 4 | 0.1119 |
| TP53wt | MYCNwt | 13.6674 | 13.8973 | −0.23 | 8 | 8 | 0.2458 |

*UVSSAm - ATRXwt was unable to be performed due to small sample size
* NR indicates not reached
*BRCA2m-BRCA1m and BRCA2m-BRCA1wt were unable to be performed due to small sample size
*MUTYHm - BRCA2m, MUTYHm - BRCA2wt, MUTYHwt - BRCA2m were unable to be performed due to small sample size
* PIK3CAm - BRCA2m, PIK3CAm - BRCA2wt, PIK3CAwt - BRCA2m were unable to be performed due to small sample size
* PRKNm - BRCA2m, PRKNm - BRCA2wt, PRKNwt - BRCA2m were unable to be performed due to small sample size
* RAD51m - BRCA2m, RAD51m - BRCA2wt, RAD51wt - BRCA2m were unable to be performed due to small sample size
* RUNX1m - BRCA2m, RUNX1m - BRCA2wt, RUNX1wt - BRCA2m were unable to be performed due to small sample size
* TP53m - ERCC2m and TP53wt - ERCC2m were unable to be performed due to small sample size
*TP53wt - HTR2Cm was unable to be performed due to small sample size
* NR indicates not reached

TABLE 6
RFS from procurement - small distance gene pairs

| | | Vigil ® | Placebo | | | | |
|---|---|---|---|---|---|---|---|
| Gene 1 | Gene 2 | Median | Median | Difference | nVigil | nPlacebo | PVAL |
| BRCA1m | ARID1Awt | 17.577 | 16 | 1.577 | 5 | 6 | 0.4216 |
| BRCA1wt | ARID1Am | 17.9713 | 14.3573 | 3.614 | 3 | 3 | 0.0294 |
| BRCA1wt | ARID1Awt | 19.3511 | 15.8686 | 3.4825 | 39 | 34 | 0.1117 |
| BRCA2wt | ARID1Am | 17.9713 | 14.3573 | 3.614 | 3 | 3 | 0.0294 |
| BRCA2wt | ARID1Awt | 18.3326 | 15.8686 | 2.4641 | 43 | 33 | 0.0476 |
| CTNNB1wt | ARID1Am | 17.9713 | 14.3573 | 3.614 | 3 | 4 | 0.0365 |
| CTNNB1wt | ARID1Awt | 18.2998 | 16 | 2.2998 | 42 | 40 | 0.2056 |
| ERCC2wt | ARID1Am | 17.9713 | 14.3573 | 3.614 | 3 | 4 | 0.0365 |
| ERCC2wt | ARID1Awt | 18.3326 | 16 | 2.3326 | 44 | 37 | 0.0853 |
| HTR2Cm | ARID1Awt | 18.2998 | 17.7741 | 0.5257 | 17 | 5 | 0.2549 |
| HTR2Cwt | ARID1Am | 17.9713 | 14.3573 | 3.614 | 3 | 4 | 0.0365 |
| HTR2Cwt | ARID1Awt | 25.4949 | 16 | 9.4949 | 27 | 35 | 0.1505 |
| MUTYHwt | ARID1Am | 17.9713 | 14.3573 | 3.614 | 3 | 4 | 0.0365 |
| MUTYHwt | ARID1Awt | 19.3511 | 16 | 3.3511 | 42 | 40 | 0.1117 |
| NF1wt | ARID1Am | 17.9713 | 14.3573 | 3.614 | 3 | 4 | 0.0365 |
| NF1wt | ARID1Awt | 18.3326 | 16 | 2.3326 | 44 | 38 | 0.0971 |
| PIK3CAwt | ARID1Am | 17.9713 | 14.3573 | 3.614 | 3 | 4 | 0.0365 |
| PIK3CAwt | ARID1Awt | 18.3326 | 16 | 2.3326 | 43 | 39 | 0.1043 |
| PRKNwt | ARID1Am | 17.9713 | 14.3573 | 3.614 | 3 | 4 | 0.0365 |
| PRKNwt | ARID1Awt | 18.3326 | 16 | 2.3326 | 42 | 39 | 0.1065 |
| RAD51Cwt | ARID1Am | 17.9713 | 14.3573 | 3.614 | 3 | 4 | 0.0365 |
| RAD51Cwt | ARID1Awt | 19.3511 | 17.7741 | 1.577 | 43 | 39 | 0.1094 |
| RUNX1wt | ARID1Am | 17.9713 | 14.3573 | 3.614 | 3 | 4 | 0.0365 |
| RUNX1wt | ARID1Awt | 19.3511 | 16 | 3.3511 | 43 | 39 | 0.0942 |
| UVSSAm | ARID1Awt | 17.577 | 17.7741 | −0.1971 | 11 | 11 | 0.1999 |
| UVSSAwt | ARID1Am | 17.9713 | 14.3573 | 3.614 | 3 | 3 | 0.0294 |
| UVSSAwt | ARID1Awt | 19.3511 | 15.8686 | 3.4825 | 33 | 29 | 0.1786 |
| BRCA2wt | ATRXm | 18.3326 | 15.8686 | 2.4641 | 40 | 28 | 0.1386 |
| BRCA2wt | ATRXwt | 17.9713 | 12.3532 | 5.6181 | 6 | 8 | 0.0373 |
| NF1wt | ATRXm | 18.3326 | 16 | 2.3326 | 41 | 33 | 0.2374 |
| NF1wt | ATRXwt | 17.9713 | 12.4846 | 5.4867 | 6 | 9 | 0.0505 |
| UVSSAm | ATRXm | NR | 18.7269 | NR | 9 | 6 | 0.1777 |
| UVSSAwt | ATRXm | 18.3326 | 14.9158 | 3.4168 | 32 | 28 | 0.3346 |
| UVSSAwt | ATRXwt | NR | 15.1786 | NR | 4 | 4 | 0.0386 |
| BRCA2wt | BRCA1m | 17.577 | 16 | 1.577 | 5 | 6 | 0.4216 |
| BRCA2wt | BRCA1wt | 18.3326 | 14.7844 | 3.5483 | 41 | 30 | 0.0274 |
| ERCC2wt | BRCA1m | 17.577 | 16 | 1.577 | 5 | 7 | 0.4765 |
| ERCC2wt | BRCA1wt | 18.3326 | 14.9158 | 3.4168 | 42 | 34 | 0.0595 |
| ERCC2wt | BRCA2wt | 18.2998 | 14.9158 | 3.384 | 46 | 34 | 0.0334 |
| HTR2Cm | BRCA2wt | 18.2998 | 17.7741 | 0.5257 | 17 | 5 | 0.2549 |
| HTR2Cwt | BRCA2wt | 19.6468 | 14.9158 | 4.731 | 29 | 31 | 0.0466 |
| MUTYHwt | BRCA2wt | 18.3326 | 14.9158 | 3.4168 | 44 | 36 | 0.0302 |
| MYCNm | BRCA2wt | 17.347 | 15.9343 | 1.4127 | 13 | 12 | 0.185 |
| MYCNwt | BRCA2wt | 18.2998 | 14.7844 | 3.5154 | 33 | 24 | 0.0591 |
| NF1wt | BRCA2wt | 18.2998 | 14.9158 | 3.384 | 46 | 35 | 0.0396 |
| OGG1wt | BRCA2wt | 18.2998 | 14.9158 | 3.384 | 45 | 35 | 0.033 |
| PIK3CAwt | BRCA2wt | 18.2998 | 14.9158 | 3.384 | 45 | 35 | 0.0213 |
| PRKNwt | BRCA2wt | 18.2998 | 14.9158 | 3.384 | 44 | 35 | 0.0227 |
| RAD51Cwt | BRCA2wt | 18.3326 | 15.8686 | 2.4641 | 45 | 35 | 0.029 |
| RUNX1wt | BRCA2wt | 18.3326 | 14.9158 | 3.4168 | 45 | 35 | 0.0241 |
| NF1wt | ERCC2wt | 18.3326 | 15.8686 | 2.4641 | 47 | 39 | 0.0516 |
| PIK3CAwt | ERCC2wt | 18.2998 | 15.8686 | 2.4312 | 46 | 40 | 0.0555 |
| PRKNwt | ERCC2wt | 18.3326 | 15.8686 | 2.4641 | 45 | 40 | 0.0573 |
| RUNX1wt | ERCC2wt | 18.3326 | 15.8686 | 2.4641 | 46 | 40 | 0.0536 |
| TP53m | ERCC2wt | 19.3511 | 15.8686 | 3.4825 | 33 | 30 | 0.0408 |
| TP53wt | ERCC2wt | 18.2998 | 25.5934 | −7.2936 | 14 | 11 | 0.4641 |
| TP53m | MYCNm | NR | 15.9343 | NR | 7 | 8 | 0.0529 |
| TP53m | MYCNwt | 17.9713 | 17.7741 | 0.1971 | 26 | 24 | 0.2649 |
| TP53wt | MYCNm | 13.963 | 20.2218 | −6.2587 | 6 | 4 | 0.177 |
| TP53wt | MYCNwt | 19.6468 | 14.9158 | 4.731 | 8 | 8 | 0.195 |
| TP53m | NF1wt | 19.3511 | 16 | 3.3511 | 33 | 30 | 0.0577 |
| TP53wt | NF1wt | 18.2998 | 14.9158 | 3.384 | 14 | 12 | 0.4102 |

*BRCA1m - ARID1Am was unable to be performed due to small sample size
*BRCA2m-BARID1Am and BRCA2m-ARID1Awt were unable to be performed due to small sample size
* CTNNB1m-ARID1Am and CTNNB1m-ARID1Awt were unable to be performed due to small sample size
* ERCC2m-ARID1Am and ERCC2m-ARID1Awt were unable to be performed due to small sample size
*HTR2Cm - ARID1Am was unable to be performed due to small sample size
* MUTYHm-ARID1Am and MUTYHm-ARID1Awt were unable to be performed due to small sample size
* NF1m-ARID1Am and NF1m-ARID1Awt were unable to be performed due to small sample size
* PIK3CAm-ARID1Am and PIK3CAm-ARID1Awt were unable to be performed due to small sample size
* PRKNm-ARID1Am and PRKNm-ARID1Awt were unable to be performed due to small sample size
* RAD51Cm-RARID1Am and RAD51Cm-ARID1Awt were unable to be performed due to small sample size
* RUNX1m-ARID1Am and RUNX1m-ARID1Awt were unable to be performed due to small sample size
* UVSSAm-ARID1Am was unable to be performed due to small sample size
* BRCA2m-ATRXm and BRCA2m-ATRXwt were unable to be performed due to small sample size
* NF1m-ATRXm and NF1m-ATRXwt were unable to be performed due to small sample size
* UVSSAm-ATRXwt was unable to be performed due to small sample size
*BRCA2m-BRCA1m and BRCA2m-BRCA1wt were unable to be performed due to small sample size
* ERCC2m-BRCA1m and ERCC2m-BRCA1wt were unable to be performed due to small sample size
*ERCC2m - BRCA2m, ERCC2m - BRCA2wt, ERCC2wt - BRCA2m were unable to be performed due to small sample size
*HTR2Cm-BRCA2m and HTR2Cwt-BRCA2m were unable to be performed due to small sample size
*MUTYHm - BRCA2m, MUTYHm - BRCA2wt, MUTYHwt - BRCA2m were unable to be performed due to small sample size
*MYCNm - BRCA2m and MYCN- BRCA2m were unable to be performed due to small sample size
* NF1m - BRCA2m, NF1m - BRCA2wt, NF1wt - BRCA2m were unable to be performed due to small sample size
* OGG1m - BRCA2m, OGG1m - BRCA2wt, OGG1wt - BRCA2m were unable to be performed due to small sample size
* PIK3CAm - BRCA2m, PIK3CAm - BRCA2wt, PIK3CAwt - BRCA2m were unable to be performed due to small sample size
* PRKNm - BRCA2m, PRKNm - BRCA2wt, PRKNwt - BRCA2m were unable to be performed due to small sample size
* RAD51Cm - BRCA2m, RAD51Cm - BRCA2wt, RAD51Cwt - BRCA2m were unable to be performed due to small sample size
* RUNX1m - BRCA2m, RUNX1m - BRCA2wt, RUNX1wt - BRCA2m were unable to be performed due to small sample size
* NF1m - ERCC2m, NF1m - ERCC2wt, NF1wt - ERCC2m were unable to be performed due to small sample size
* PIK3CAm - ERCC2m, PIK3CAm - ERCC2wt, PIK3CAwt - ERCC2m were unable to be performed due to small sample size
* PRKNm - ERCC2m, PRKNm - ERCC2wt, PRKNwt - ERCC2m were unable to be performed due to small sample size
* RUNX1m - ERCC2m, RUNX1m - ERCC2wt, RUNX1wt - ERCC2m were unable to be performed due to small sample size
* TP53m - ERCC2m, TP53wt- ERCC2m were unable to be performed due to small sample size
* NR indicates not reached
* TP53m - NF1m, TP53wt- NF1m were unable to be performed due to small sample size

TABLE 7
RFS from procurement - hub genes

| Gene | VigilMedian | PlaceboMedian | Difference | nVigil | nPlacebo | PVAL |
|---|---|---|---|---|---|---|
| PIK3CAwt | 18.2998 | 15.8686 | 2.4312 | 46 | 43 | 0.0799 |
| NF1wt | 18.3326 | 16 | 2.3326 | 47 | 42 | 0.0742 |
| BRCA2wt | 18.2998 | 14.9158 | 3.384 | 46 | 36 | 0.0304 |
| ARID1Am | 17.9713 | 14.3573 | 3.614 | 3 | 4 | 0.0365 |
| ARID1Awt | 18.3326 | 16 | 2.3326 | 44 | 40 | 0.1142 |
| MUTYHwt | 18.3326 | 16 | 2.3326 | 45 | 44 | 0.0879 |

* PIK3CAm was unable to be performed due to small sample size
* NF1m was unable to be performed due to small sample size
* BRCA2m was unable to be performed due to small sample size
* MUTYHm was unable to be performed due to small sample size

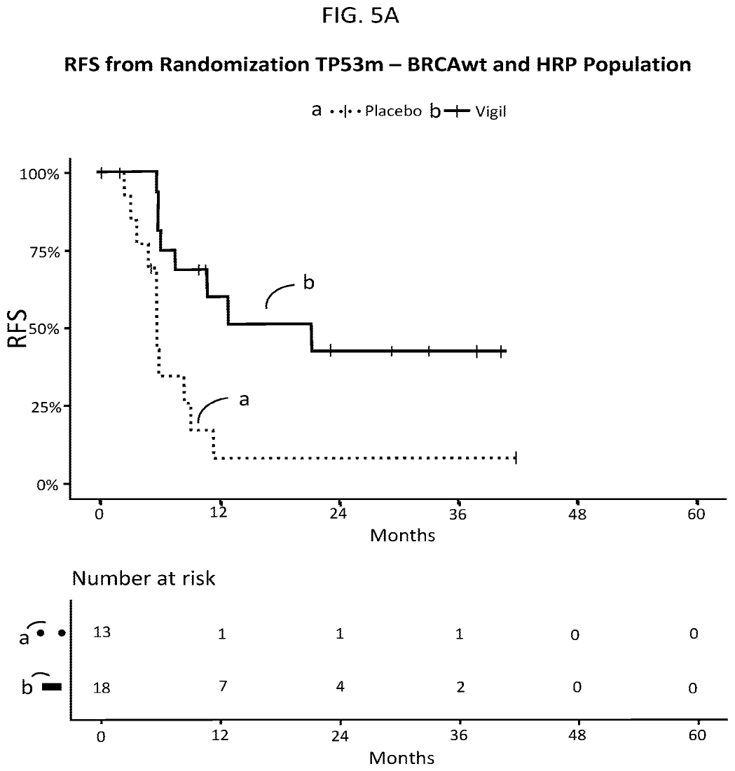

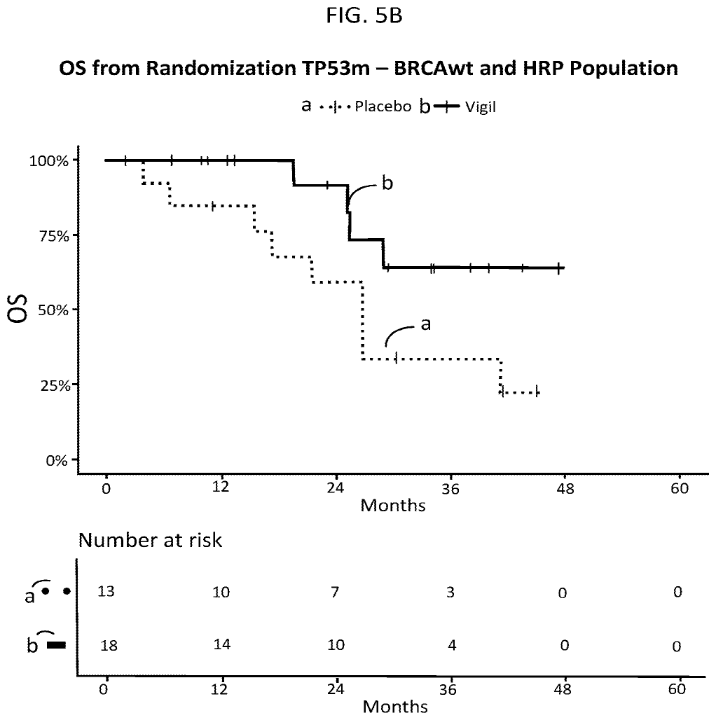

Homologous Recombination Status. KM analysis was conducted to determine the effect of homologous recombination status on the TP53m-BRCAwt population. A score of <42, as defined by Myriad Genetics was used to identify patients who were HRP and a score of ≥42 indicated patients were HRD. RFS in the TP53m-BRCAwt and HRP group was improved to 21.1 vs. 5.6 months (HR=0.26, p=0.001) in Vigil® vs. placebo patients (FIG. 5A). OS was also improved in HRP and TP53m-BRCAwt patients from randomization. In the Vigil® treated group, OS was not reached while placebo was 27.0 months (HR=0.33, p=0.02) (FIG. 5B).

### Discussion

Our network based analysis of pathogenic gene mutations points us towards potentially an optimal population, specifically patients with a HRP malignant cell profile including BRCAwt and TP53 mutant gene signals, who from Vigil® based therapy as related to RFS and OS advantage. These results are only hypothesis generating, but suggest a novel approach to optimizing a target population for therapy with Vigil. In this approach, the STRING database was utilized to construct an unbiased network to describe the functional similarity between genes, thereby providing mechanistic understanding of the potential effect of wild type or mutant variants. This approach circumvents a potential limitation of DNA variant data and may provide for target population identification more effectively given our current limited understanding of comprehensive molecular signal expression pathways and relationship to clinical benefit impact. In this manner approach, one can describe the genes of high importance by computationally analyzing properties of the malignant network, such as the topological distance between genes, C-scores and hub genes. Through these analyses in the HRP ovarian population treated with Vigil, three gene variants stood out across all analytic methods: BRCA1, BRCA2, and TP53. Similar methodology has been used by others involving various cancer types and have demonstrated molecular collaboration with clinical outcomes [32, 33, 34].

The combination of STRING-generated topological distance and sample-derived probability of co-mutation is manifested as the C-score for two genes, and the Cumulative C-score (cumC-score) is the aggregate of one gene's interaction with every other gene in the network. Gene pairs identified by C-scores often involve central cancer genes which correlate with increased tumorigenesis and sensitivity/resistance to anticancer therapeutics [35]. For genes that are both highly represented in our sample and in the literature, the cumC-score value is both robust and nuanced, as the numerator is derived from patient data and the denominator is generated from current literature. Here, we identified TP53, BRCA1, and BRCA2 as genes with the highest cumC-scores of the genes present in patient samples. The high cumC-score suggests that particular variants of these genes correspond to drug response, as cumC-scores correlate with sensitivity and resistance [35]. Indeed, we found that TP53m and BRCAwt correlated with increased RFS and OS benefit to Vigil®.

Analysis of hub genes similarly identified BRCA and TP53 as central ovarian cancer genes. Previous work demonstrated that hub gene data provided clinical insight to differences in OS. In one previous study, four of 16 identified hub genes in the studied sample (CCNB1, CENPF, KIF11, and ZWINT) were associated with decreased OS of patients with ovarian cancer [36]. Authors of this study posit that mutations in these hub genes, which occupy the intersection of many cellular pathways, results in rippling dysregulation of numerous cellular functions. Thus, by altering a single hub gene, cellular homeostasis may be impacted on a larger scale. This disruption may be associated with tumor progression, immune inhibition, and any number of cancer hallmarks, which may explain the association of hub genes with a poor prognosis [37, 38]. The hub gene analysis presented in our paper identified TP53m, BRCA1wt, BRCA2 wt as core hub genes with RFS advantage in Vigil® treated patients, potentially indicating a broader genetic network for target population of Vigil®. Moreover, this approach supports a strategic shift in targeted therapeutic development towards targeting related network genomic variants.

Our results also support that the pathways impacted by BRCA must be intact for Vigil® to function optimally, while the pathways impacted by TP53 may be dysregulated. Similarly, the integrity of the homologous repair pathway and its associated genes (HRP genotype) may also be important for optimal Vigil® results. This suggests a cancer homeostasis formed by the combination of gene variants that creates an optimal environment for drug sensitivity or resistance. In our case, we hypothesize that the interaction of pathways generated by functional HR or BRCA proteins and disrupted TP53 protein creates the ideal molecular setting for Vigil® therapy.

These results involving Vigil® immunotherapy which has a different mechanism of action from immune checkpoint inhibitor therapy however are slightly counterintuitive, to target populations involved in checkpoint inhibitor based immunotherapy, as current theory would posit that deficiencies in DNA repair would yield more tumor neoantigens and higher TMB [39]. Intratumoral heterogeneity (ITH) associated with high TMB provides an increase in variation of neoepitopes between cells within a tumor and can dilute a consolidated immune response against lower frequency clonal neoantigens [28, 30]. As related to Vigil® mechanism of action a more robust immune attack is mounted against clonal neoepitopes displayed by the majority of cancer cells in a low ITH environment. TP53m is associated with increased TMB and tumor aneuploidy level (TAL), which have conflicting impacts on immune responsivity. TMB tends to correlate with sensitivity to certain immunotherapies particularly checkpoint inhibitors [39, 40]. TAL is the degree of chromosomal mis-segregation, and is associated with poor response to immunotherapy [41, 42]. TP53's impact on immunogenicity is also tissue dependent, which may be determined by differential gene expression within tissue types [42-44]. This phenomenon may further explain mutant TP53 genotype's association with improved response to Vigil® immunotherapy in the ovarian cancer BRCAwt population. We did not observe independent effect of benefit to high TMB with Vigil. Although previously reported benefit in RFS and OS in BRCAwt patients was not adversely effected by high TMB [28].

In conclusion, we demonstrated proof of support for use of DNA analytic methods to separate resistant and sensitive populations to Vigil®. These techniques create a robust approach to analyze how the nodal network relationship between genes affects clinical response to Vigil® when used as maintenance therapy in advanced Stage III/IV resectable disease patients. These results further support novel use of network based analysis to identify other more sensitive gene targets and potentially additional novel targeted therapeutic combinations with Vigil® and possibly other immunotherapeutics.

### REFERENCES

1. Siegel, R. L., K. D. Miller, and A. Jemal, Cancer statistics, 2019. CA Cancer J Clin, 2019. 69(1): p. 7-34.
2. Bray, F., et al., Global cancer statistics 2018: GLOBOCAN estimates of incidence and mortality worldwide for 36 cancers in 185 countries. CA Cancer J Clin, 2018. 68(6): p. 394-424.
3. Cobum, S. B., et al., International patterns and trends in ovarian cancer incidence, overall and by histologic subtype. Int J Cancer, 2017. 140(11): p. 2451-2460.
4. Momenimovahed, Z., et al., Ovarian cancer in the world: epidemiology and risk factors. Int J Womens Health, 2019. 11: p. 287-299.
5. Yoneda, A., et al., Breast and ovarian cancers: a survey and possible roles for the cell surface heparan sulfate proteoglycans. J Histochem Cytochem, 2012. 60(1): p. 9-21.
6. Walsh, T., et al., Mutations in 12 genes for inherited ovarian, fallopian tube, and peritoneal carcinoma identified by massively parallel sequencing. Proc Natl Acad Sci USA, 2011. 108(44): p. 18032-7.
7. Toss, A., et al., Hereditary ovarian cancer: not only BRCA 1 and 2 genes. Biomed Res Int, 2015. 2015: p. 341723.
8. Cortez, A. J., et al., Advances in ovarian cancer therapy. Cancer Chemother Pharmacol, 2018. 81(1): p. 17-38.
9. Jelovac, D. and D. K. Armstrong, Recent progress in the diagnosis and treatment of ovarian cancer. CA Cancer J Clin, 2011. 61(3): p. 183-203.
10. Foley, O W., J. A. Rauh-Hain, and M. G. del Carmen, Recurrent epithelial ovarian cancer: an update on treatment. Oncology (Williston Park), 2013. 27(4): p. 288-94, 298.
11. Hanker, L. C., et al., The impact of second to sixth line therapy on survival of relapsed ovarian cancer after primary taxane/platinum-based therapy. Ann Oncol, 2012. 23(10): p. 2605-12.
12. Ledermann, J., et al., Olaparib maintenance therapy in platinum-sensitive relapsed ovarian cancer. N Engl J Med, 2012. 366(15): p. 1382-92.
13. Gibson, B. A. and W. L. Kraus, New insights into the molecular and cellular functions of poly(ADP-ribose) and PARPs. Nat Rev Mol Cell Biol, 2012. 13(7): p. 411-24.
14. Faraoni, I. and G. Graziani, Role of BRCA Mutations in Cancer Treatment with Poly(ADP-ribose) Polymerase (PARP) Inhibitors. Cancers (Basel), 2018. 10(12): p. 487-507.
15. Kaelin, W. G., Jr., The concept of synthetic lethality in the context of anticancer therapy. Nat Rev Cancer, 2005. 5(9): p. 689-98.
16. Chan, N., et al., Contextual synthetic lethality of cancer cell kill based on the tumor microenvironment. Cancer Res, 2010. 70(20): p. 8045-54.
17. González-Martin, A., et al., Niraparib in Patients with Newly Diagnosed Advanced Ovarian Cancer. New England Journal of Medicine, 2019. 381(25): p. 2391-2402.
18. Ray-Coquard, I., et al., Olaparib plus Bevacizumab as First-Line Maintenance in Ovarian Cancer. N Engl J Med, 2019. 381(25): p. 2416-2428.
19. Senzer, N., et al., Long Term Follow Up: Phase I Trial of “bi-shRNA furin/GMCSF DNA/Autologous Tumor Cell” Immunotherapy (FANG™) in Advanced Cancer. Journal of Vaccines & Vaccination, 2013. 4(8): p. 209.
20. Oh, J., et al., Phase II study of Vigil® DNA engineered immunotherapy as maintenance in advanced stage ovarian cancer. Gynecol Oncol, 2016. 143(3): p. 504-510.
21. Herron, J., et al., Vigil: Personalized Immunotherapy Generating Systemic Cytotoxic T cell Response. Cancer Science and Research, 2020. 3(1): p. 1-4.
22. Rocconi, R. P., et al., Phase I Study of Autologous Gemogenovatucel-T: Long Term Follow up on Survival with Evaluation of Molecular Signals of Immune Response in Recurrent Ovarian Cancer. BMJ Open, 2020a(Submitted).
23. Barve, M., et al., Follow-up of bi-shRNA furin/GM-CSF Engineered Autologous Tumor Cell (EATC) Immunotherapy Vigil® inpatients with advanced melanoma. Biomedical Genetics and Genomics, 2016. 1(4).
24. Ghisoli, M., et al., Three-year Follow up of GMCSF/bi-shRNA(furin) DNA—transfected Autologous Tumor Immunotherapy (Vigil) in Metastatic Advanced Ewing's Sarcoma. Mol Ther, 2016. 24(8): p. 1478-83.
25. Ghisoli, M., et al., Pilot Trial of FANG Immunotherapy in Ewing's Sarcoma. Mol Ther, 2015. 23(6): p. 1103-1109.
26. Oh, J., et al., Long-term follow-up of Phase 2A trial results involving advanced ovarian cancer patients treated with Vigil® in frontline maintenance. Gynecol Oncol Rep, 2020. 34: p. 100648.
27. Senzer, N., et al., Phase I trial of “bi-shRNAi(furin)/GMCSF DNA/autologous tumor cell” vaccine (FANG) in advanced cancer. Mol Ther, 2012. 20(3): p. 679-86.
28. Rocconi, R. P., et al., Gemogenovatucel-T (Vigil) immunotherapy as maintenance in frontline stage III/IV ovarian cancer (VITAL): a randomised, double-blind, placebo—controlled, phase 2b trial. The Lancet Oncology, 2020. 21(12): p. 1661-1672.
29. Rocconi, R. P., et al., Homologous Recombination Proficient Ovarian Cancer: The Searchfor Efficient Maintenance Therapy. Gynecologic Oncology, 2020b(Submitted).
30. McGranahan, N., et al., Clonal neoantigens elicit T cell immunoreactivity and sensitivity to immune checkpoint blockade. Science, 2016. 351(6280): p. 1463-9.
31. Rocconi, R. P., et al., Randomized Double-Blind Placebo Controlled Trial of Vigil® (Gemogenovatucel-T) Immunotherapy (VITAL study) as Maintenance in Frontline Stage III/IV Ovarian Cancer. Lancet Oncology, 2020. In press.
32. von Mering, C., et al., STRING: known and predicted protein-protein associations, integrated and transferred across organisms. Nucleic Acids Res, 2005. 33(Database issue): p. D433-7.
33. Shannon, P., et al., Cytoscape: a software environment for integrated models of biomolecular interaction networks. Genome Res, 2003. 13(11): p. 2498-504.
34. Li, B. Q., et al., Identification of colorectal cancer related genes with mRMIR and shortest path in protein-protein interaction network. PLoS One, 2012. 7(4): p. e33393.
35. Liu, C., et al., Individualized genetic network analysis reveals new therapeutic vulnerabilities in 6,700 cancer genomes. PLoS Comput Biol, 2020. 16(2): p. e1007701.
36. Xu, Z., et al., Identification of candidate biomarkers and analysis of prognostic values in ovarian cancer by integrated bioinformatics analysis. Med Oncol, 2016. 33(11): p. 130.
37. Hanahan, D. and R. A. Weinberg, Hallmarks of cancer: the next generation. Cell, 2011. 144(5): p. 646-74.
38. Hanahan, D. and R. A. Weinberg, The Hallmarks of Cancer. Cell, 2000. 100(1): p. 57-70.
39. Choucair, K., et al., TMB: a promising immune-response biomarker, and potential spearhead in advancing targeted therapy trials. Cancer Gene Ther, 2020. 27(12): p. 841-853.
40. Yarchoan, M., et al., PD-L1 expression and tumor mutational burden are independent biomarkers in most cancers. JCI Insight, 2019. 4(6).
41. Davoli, T., et al., Tumor aneuploidy correlates with markers of immune evasion and with reduced response to immunotherapy. Science, 2017. 355(6322).
42. Li, L., M. Li, and X. Wang, Cancer type-dependent correlations between TP53 mutations and antitumor immunity. DNA Repair (Amst), 2020. 88: p. 102785.
43. Andrysik, Z., et al., Identification of a core TP53 transcriptional program with highly distributed tumor suppressive activity. Genome Res, 2017. 27(10): p. 1645-1657.
44. Kastenhuber, E. R. and S. W. Lowe, Putting p53 in Context. Cell, 2017. 170(6): p. 1062-1078.

The references listed above are hereby incorporated by reference in their entireties for all purposes.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for predicting the responsiveness of a cancer in a cancer patient to a cancer treatment, comprising determining the genotypes of BRCA1 and/or BRCA2 in a sample from the cancer patient,
wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2; and
wherein a determination of one or more of the following genotypes: (1) BRCA1wt, (2) BRCA2 wt, and (3) BRCA1wt and BRCA2 wt, indicates that the cancer patient is responsive to the cancer treatment.

2. The method of claim 1, wherein the method comprises determining the genotypes of two or more genes selected from the group consisting of BRCA1, BRCA2, TP53, PIK3CA, NF1, ARID1A, MYCNOS, and MUTYH, and wherein one of the two or more genes is BRCA1 or BRCA2.

3. The method of claim 2, wherein a determination of one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt, indicates that the cancer patient is responsive to the cancer treatment.

4. The method of any one of claims 1 to 3, wherein the method comprises determining the genotypes of three genes, and wherein two of the three genes are BRCA1 and BRCA2.

5. The method of claim 4, wherein a determination of one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt, indicates that the cancer patient is response to the cancer treatment.

6. A method for predicting the responsiveness of a cancer in a cancer patient to a cancer treatment, comprising determining the genotypes of a first gene and a second gene, in a sample from the cancer patient,
wherein the cancer treatment comprises administering to the cancer patient an expression vector comprising: (a) a first insert comprising a nucleic acid sequence encoding a Granulocyte Macrophage Colony Stimulating Factor (GM-CSF) sequence; and (b) a second insert comprising a sequence according to SEQ ID NO:2;
wherein the first gene is ARID1A and the second gene is selected from the group consisting of CTNNB1, MUTYH, NF1, PIK3CA, and UVSSA; and
wherein a determination of one or more of the following pairs of genotypes: CTNNB1wt and ARID1Am, MUTYHwt and ARID1Am, NF1wt and ARID1Am, PIK3CAwt and ARID1Am, and UVSSAwt and ARID1Am, indicates that the cancer patient is responsive to the cancer treatment.

7. The method of any one of claims 1 to 6, wherein the cancer patient is identified as homologous recombination proficient.

8. The method of any one of claims 1 to 7, wherein the method, upon the determination of genotype(s) that indicates responsiveness of the cancer patient to the cancer treatment, further comprises treating the cancer patient with the cancer treatment.

9. The method of any one of claims 1 to 8, wherein the sample is a biopsy sample.

10. The method of claim 9, wherein the biopsy sample is a biopsy sample of the tumor cells or a biopsy sample of circulating tumor cells.

11. A method for treating a cancer in a cancer patient in need thereof, the method comprising administering to the cancer patient an expression vector comprising:
a) a first insert comprising a nucleic acid sequence encoding a Granulocyte Macrophage Colony Stimulating Factor (GM-CSF) sequence; and
b) a second insert comprising a sequence according to SEQ ID NO:2, wherein the cancer patient comprises one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt.

12. A method for treating a cancer in a cancer patient in need thereof, the method comprising administering to the cancer patient an expression vector comprising:
a) a first insert comprising a nucleic acid sequence encoding a Granulocyte Macrophage Colony Stimulating Factor (GM-CSF) sequence; and
b) a second insert comprising a sequence according to SEQ ID NO:2,
wherein the cancer patient comprises one or more of the following triplets of genotypes: TP53m, BRCA1wt, and BRCA2 wt; BRCA1wt, BRCA2 wt, and PIK3CAwt;
BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt.

13. A method for treating a cancer in a cancer patient in need thereof, the method comprising administering to the cancer patient an expression vector comprising:
a) a first insert comprising a nucleic acid sequence encoding a Granulocyte Macrophage Colony Stimulating Factor (GM-CSF) sequence; and
b) a second insert comprising a sequence according to SEQ ID NO:2,
wherein the cancer patient comprises one or more of the following pairs of genotypes: CTNNB1 wt and ARID1Am, MUTYHwt and ARID1Am, NF1 wt and ARID1Am, PIK3CAwt and ARID1Am, and UVSSAwt and ARID1Am, indicates that the cancer patient is responsive to the cancer treatment.

14. The method of any one of claims 11 to 13, wherein the cancer patient is identified as homologous recombination proficient.

15. A method for treating a cancer in a cancer patient in need thereof, the method comprising:
1) genotyping the cancer patient to identify genotypes comprising BRCA1wt and/or BRCA2 wt in a sample from the cancer patient;
2) administering to the cancer patient an expression vector comprising:
a) a first insert comprising a nucleic acid sequence encoding a Granulocyte Macrophage Colony Stimulating Factor (GM-CSF) sequence; and
b) a second insert comprising a sequence according to SEQ ID NO:2, to thereby treat the cancer patient.

16. The method of claim 15, wherein step 1) further comprises one or more of the following pairs of genotypes: TP53m and BRCA1wt; TP53m and BRCA2 wt; BRCA1wt and PIK3CAwt; BRCA1wt and NF1wt; BRCA1wt and ARID1Awt; BRCA1wt and MYCNOSwt; BRCA1wt and MUTYHwt; BRCA2 wt and PIK3CAwt; BRCA2 wt and NF1wt; BRCA2 wt and ARID1Awt; BRCA2 wt and MYCNOSwt; and BRCA2 wt and MUTYHwt.

17. The method of claim 15 or 16, wherein step 1) further comprises one or more of the following triplets of genotypes: TP53m, BRCA1wt, and BRCA2 wt; BRCA1wt, BRCA2 wt, and PIK3CAwt; BRCA1wt, BRCA2 wt, and NF1wt; BRCA1wt, BRCA2 wt, and ARID1Awt; BRCA1wt, BRCA2 wt, and MYCNOSwt; and BRCA1wt, BRCA2 wt, and MUTYHwt.

18. A method for treating a cancer in a cancer patient in need thereof, the method comprising:
1) genotyping the cancer patient to identify one or more of the following pairs of genotypes: CTNNB1 wt and ARID1Am, MUTYHwt and ARID1Am, NF1 wt and ARID1Am, PIK3CAwt and ARID1Am, and UVSSAwt and ARID1Am, in a sample from the cancer patient;
2) administering to the cancer patient an expression vector comprising:
a) a first insert comprising a nucleic acid sequence encoding a Granulocyte Macrophage Colony Stimulating Factor (GM-CSF) sequence; and
b) a second insert comprising a sequence according to SEQ ID NO:2, to thereby treat the cancer patient.

19. The method of any one of claims 15 to 18, wherein the sample is a biopsy sample.

20. The method of claim 19, wherein the biopsy sample is a biopsy sample of the tumor cells or a biopsy sample of circulating tumor cells.

21. The method of any one of claims 1 to 20, wherein the GM-CSF is a human GM-CSF sequence.

22. The method of any one of claims 1 to 21, wherein the expression vector further comprises a promoter.

23. The method of claim 22, wherein the promoter is a cytomegalovirus (CMV) mammalian promoter.

24. The method of any one of claims 1 to 23, wherein the expression vector further comprises a CMV enhancer sequence and a CMV intron sequence.

25. The method of any one of claims 1 to 24, wherein the expression vector further comprises a nucleic acid sequence encoding a picomaviral 2A ribosomal skip peptide between the first and the second nucleic acid inserts.

26. The method of any one of claims 1 to 25, wherein the expression vector is within an autologous cancer cell that is transfected with the expression vector.

27. The method of claim 26, wherein the autologous cancer cell is administered to the individual as a dose of about 1×10⁶ cells to about 5×10⁷ cells.

28. The method of claim 26 or 27, wherein the autologous cancer cell is administered to the individual once a month.

29. The method of any one of claims 26 to 28, wherein the autologous cancer cell is administered to the individual from 1 to 12 months.

30. The method of any one of claims 26 to 29, wherein the autologous cancer cell is administered to the cancer patient by intradermal injection.

31. The method of any one of claims 1 to 30, wherein the first insert and the second insert are operably linked to the promoter.

32. The method of any one of claims 1 to 31, wherein the cancer is an HRD-negative, wild-type BRCA1/2 cancer.

33. The method of any one of claims 1 to 32, wherein the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer.

34. The method of claim 33, wherein the solid tumor cancer is selected from the group consisting of endometrial cancer, biliary cancer, bladder cancer, liver hepatocellular carcinoma, gastric/esophageal cancer, ovarian cancer, melanoma, breast cancer, pancreatic cancer, colorectal cancer, glioma, non-small-cell lung carcinoma, prostate cancer, cervical cancer, kidney cancer, thyroid cancer, a neuroendocrine cancer, small cell lung cancer, a sarcoma, head and neck cancer, brain cancer, clear cell renal cell carcinoma, skin cancer, endocrine tumor, thyroid cancer, tumor of unknown origin, and a gastrointestinal stromal tumor.

35. The method of claim 33, wherein the cancer is ovarian cancer.

36. The method of claim 33, wherein the cancer is breast cancer.

37. The method of claim 33, wherein the cancer is melanoma.

38. The method of claim 33, wherein the cancer is lung cancer.

39. The method of any one of claims 1 to 33, wherein the cancer is ovarian cancer and wherein the method prevents or delays relapse of a substantially eradicated ovarian cancer.

40. The method of claim 39, wherein the substantially eradicated ovarian cancer is Stage III or Stage IV ovarian cancer.

41. The method of any one of claims 1 to 40, wherein the cancer patient received an initial therapy.

42. The method of claim 41, wherein the initial therapy comprises debulking surgery, chemotherapy, or the combination thereof.

43. The method of claim 42, wherein the chemotherapy comprises administering a platinum-based drug and a taxane.

44. The method of claim 43, wherein the platinum-based drug comprises carboplatin.

45. The method of claim 43, wherein the taxane comprises paclitaxel.

46. The method of any one of claims 1 to 45, further comprising administering an additional therapeutic agent.

47. The method of claim 46, wherein the additional therapeutic agent is a member selected from the group consisting of an angiogenesis inhibitor, a PARP inhibitor, and a checkpoint inhibitor to the individual.
